(19) United States
(12) Patent Application Publication
(10) Pub. No.: US 2026/0079124 A1
(43) Pub. Date: Mar. 19, 2026

(54) SENSOR PACKAGE AND SENSOR PACKAGE MANUFACTURING METHOD

(71) Applicant: Nuvoton Technology Corporation Japan, Kyoto (JP)

(72) Inventors: Hikari SANO, Kyoto (JP); Yukitoshi OTA, Kyoto (JP); Noriyuki NAGAI, Kyoto (JP); Satoshi KITAMURA, Kyoto (JP)

(21) Appl. No.: 19/346,730

(22) Filed: Oct. 1, 2025

Related U.S. Application Data
(63) Continuation of application No. PCT/JP2024/014163, filed on Apr. 5, 2024.
(60) Provisional application No. 63/495,661, filed on Apr. 12, 2023.

(51) Int. Cl. G01N 27/12; G01N 33/00

(52) U.S. Cl. CPC G01N 27/125; G01N 33/005

(57) ABSTRACT

A sensor package includes: a chip including a surface on which the exposed portion of a sensor unit is provided; a substrate including a surface on which the chip is mounted; and a molded resin portion covering the surface of the substrate and the surface of the chip excluding the exposed portion. The molded resin portion includes an aperture positioned above the exposed portion. The chip includes a flat part positioned outside of the exposed portion on the surface of the chip. The edge of the aperture on the surface side of the chip is formed along the flat part.

### CROSS REFERENCE TO RELATED APPLICATIONS

This is a continuation application of PCT International Patent Application No. PCT/JP2024/014163 filed on Apr. 5, 2024, designating the United States of America, which is based on and claims priority of U.S. Provisional Patent Application No. 63/495,661 filed on Apr. 12, 2023. The entire disclosures of the above-identified applications, including the specifications, drawings and claims are incorporated herein by reference in their entirety.

### FIELD

The present disclosure relates to sensor packages and sensor package manufacturing methods.

### BACKGROUND

Conventional sensor packages with sensors are known. PTL 1discloses, in FIG. 6, a sensor device with a chip, a lead frame, a bonding wire, a package, etc., having a detection structure.

### CITATION LIST

### Patent Literature

PTL 1: Japanese Unexamined Patent Application Publication No. 2010-50452

### SUMMARY

### Technical Problem

The sensor device disclosed in PTL 1 has a problem that the quality of the sensor unit is degraded.

The present disclosure provides a sensor package or the like that can suppress deterioration in the quality of the sensor unit.

### Solution to Problem

A sensor package according to one aspect of the present disclosure includes: a chip including a surface on which the exposed portion of a sensor unit is provided; a substrate including a surface on which the chip is mounted; and a molded resin portion covering the surface of the substrate and the surface of the chip excluding the exposed portion. The molded resin portion includes an aperture positioned above the exposed portion. The chip includes a flat part positioned outside of the exposed portion on the surface of the chip. The edge of the aperture on the surface side of the chip is formed along the flat part.

A sensor package according to one aspect of the present disclosure includes: a chip including a surface on which the exposed portion of a sensor unit is provided; a substrate including a surface on which the chip is mounted; and a molded resin portion covering the surface of the substrate and the surface of the chip excluding the exposed portion. The molded resin portion includes an aperture positioned above the exposed portion. The edge of the aperture on the surface side of the chip is positioned on the same plane on the surface side of the chip.

A sensor package manufacturing method according to one aspect of the present disclosure includes: forming a flat part on a surface of a chip; mounting the chip on a surface of a substrate; and arranging the protrusion of a mold above the exposed portion of a sensor unit of the chip, and forming a molded resin portion to cover the surface of the substrate and the surface of the chip excluding the exposed portion. In the forming of the flat part, the flat part is provided outside of an area where the exposed portion is formed. In the forming of the molded resin portion, the mold is arranged so that the edge of the protrusion is positioned on the flat part, to form the molded resin portion.

### Advantageous Effects

According to the present disclosure, it is possible to suppress deterioration in the quality of the sensor unit of a sensor package.

### BRIEF DESCRIPTION OF DRAWINGS

These and other advantages and features will become apparent from the following description thereof taken in conjunction with the accompanying Drawings, by way of non-limiting examples of embodiments disclosed herein.

FIG. 1 is a cross-sectional view illustrating a sensor package according to Comparative Example 1.

FIG. 2 is a diagram illustrating, for instance, the protrusion of a mold for use in resin molding of the sensor package according to Comparative Example 1.

FIG. 3 is a diagram illustrating the topmost wiring and topmost insulating film of a chip of a sensor package according to Comparative Example 2.

FIG. 4 is a diagram illustrating a cross section taken at the line IV-IV in FIG. 3.

FIG. 5 is a diagram illustrating examples of a problem that occurs in the sensor package according to Comparative Example 2.

FIG. 6 is a schematic diagram of a sensor package according to Embodiment 1.

FIG. 7 is a diagram illustrating a cross section of the chip and molded resin portion of the sensor package according to Embodiment 1.

FIG. 8 is the top view of the flat part of the chip according to Embodiment 1.

FIG. 9 is a diagram illustrating another example of the flat part of the chip according to Embodiment 1.

FIG. 10 is a diagram illustrating a cross section of the sensor unit of the sensor package according to Embodiment 1.

FIG. 11 is a flowchart illustrating a sensor package manufacturing method according to Embodiment 1.

FIG. 12 is a diagram illustrating a cross section of the chip and molded resin portion of a sensor package according to Variation 1 of Embodiment 1.

FIG. 13 is a diagram illustrating a cross section of the chip and molded resin portion of a sensor package according to Variation 2 of Embodiment 1.

FIG. 14 is the top view of the flat parts of the chip according to Variation 2 of Embodiment 1.

FIG. 15 is a diagram illustrating another example of the flat parts of the chip according to Variation 2 of Embodiment 1.

FIG. 16 is a diagram illustrating a cross section of the chip and molded resin portion of a sensor package according to Variation 3 of Embodiment 1.

FIG. 17 is a diagram illustrating a cross section of the chip and molded resin portion of a sensor package according to Variation 4 of Embodiment 1.

FIG. 18 is a diagram illustrating a cross section of the chip and molded resin portion of a sensor package according to Variation 5 of Embodiment 1.

FIG. 19 is a diagram illustrating a cross section of the chip and molded resin portion of a sensor package according to Variation 6 of Embodiment.

FIG. 20 is the top view of the flat part of the chip according to Variation 6 of Embodiment 1.

FIG. 21 is a diagram illustrating a cross section of the chip and molded resin portion of a sensor package according to Variation 7 of Embodiment 1.

FIG. 22 is the top view of the flat part of the chip according to Variation 7 of Embodiment 1.

FIG. 23 is a diagram illustrating a cross section of the chip and molded resin portion of a sensor package according to Variation 8 of Embodiment 1.

FIG. 24 is a diagram illustrating the guiding grooves of the chip according to Variation 8 of Embodiment 1.

FIG. 25 is a diagram illustrating another example of the guiding grooves of the chip according to Variation 8 of Embodiment 1.

FIG. 26 is a diagram illustrating a cross section of the chip and molded resin portion of a sensor package according to Variation 9 of Embodiment 1.

FIG. 27 is the top view of the flat parts of the chip of a sensor package according to Variation 10 of Embodiment 1.

FIG. 28 is the top view of the flat parts of the chip of a sensor package according to Variation 11 of Embodiment 1.

FIG. 29 is a diagram illustrating a cross section of the via conductors of the chip of a sensor package according to Variation 12 of Embodiment 1.

FIG. 30 is a diagram illustrating another example of a cross section of the via conductors.

FIG. 31 illustrates the top views of the via conductors.

FIG. 32 is a diagram illustrating a cross section of the chip and molded resin portion of a sensor package according to Embodiment 2.

FIG. 33 is the top view of the flat part of the chip of the sensor package according to Embodiment 2.

FIG. 34 is a diagram illustrating another example of the flat part of the chip according to Embodiment 2.

FIG. 35 is a diagram illustrating a cross section of the chip and molded resin portion of a sensor package according to Variation 1 of Embodiment 2.

FIG. 36 is a diagram illustrating a cross section of the protrusion of a mold and a sensor package according to Variation 2 of Embodiment 2.

FIG. 37 is a plan view illustrating guiding grooves in the protrusion of the mold.

FIG. 38 is a diagram illustrating another example of the guiding grooves in the protrusion of the mold.

FIG. 39 is a flowchart illustrating a sensor package manufacturing method according to Embodiment 2.

FIG. 40 is the top view of the flat part of the chip of a sensor package according to a variation of Embodiments 1 and 2.

### DESCRIPTION OF EMBODIMENTS

### Circumstances Leading to the Present Disclosure

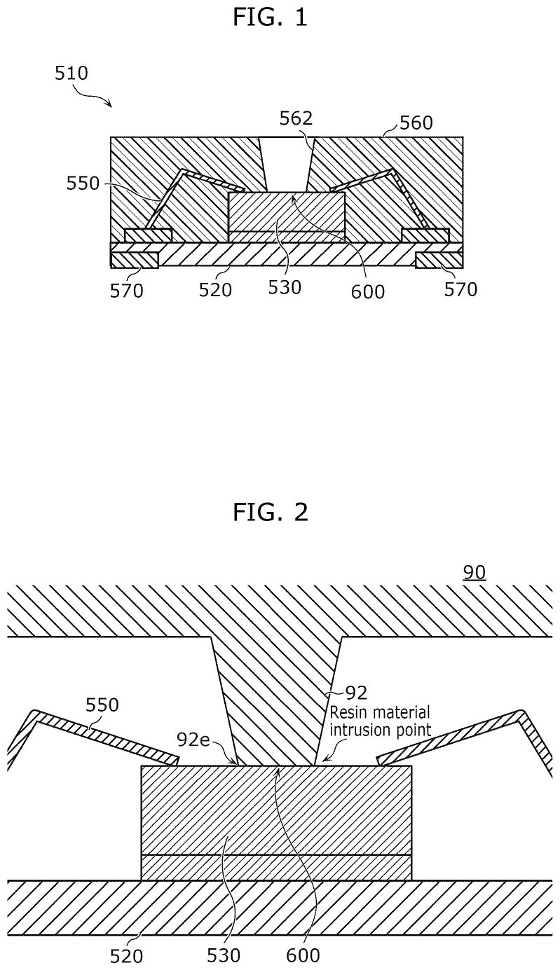

Circumstances leading to the present disclosure will be described with reference to FIG. 1 to FIG. 5.

FIG. 1 is a cross-sectional view illustrating sensor package 510 according to Comparative Example 1.

Sensor package 510 according to Comparative Example 1 includes substrate 520, chip 530 disposed on substrate 520, wires 550 that electrically connect substrate 520 and chip 530, and molded resin portion 560 formed on substrate 520 to cover wires 550 and partly cover chip 530. External terminal 570 is also illustrated in the figure.

In sensor package 510 according to Comparative Example 1, aperture 562 is formed in molded resin portion 560 to expose sensor unit 600 provided on the surface of chip 530. Aperture 562 of molded resin portion 560 is formed using protrusion 92 protruding from the inner side of mold 90.

FIG. 2 is a diagram illustrating, for instance, protrusion 92 of mold 90 for use in resin molding of sensor package 510 according to Comparative Example 1.

Molded resin portion 560 is formed, for example, through resin molding by covering substrate 520, chip 530, and wires 550 with cavity-shaped mold 90. When resin molding, a film-like resin sheet may be placed along the inner surface of mold 90. Aperture 562 is formed by injecting resin material into mold 90 with protrusion 92 of mold 90 pressed against the surface of chip 530.

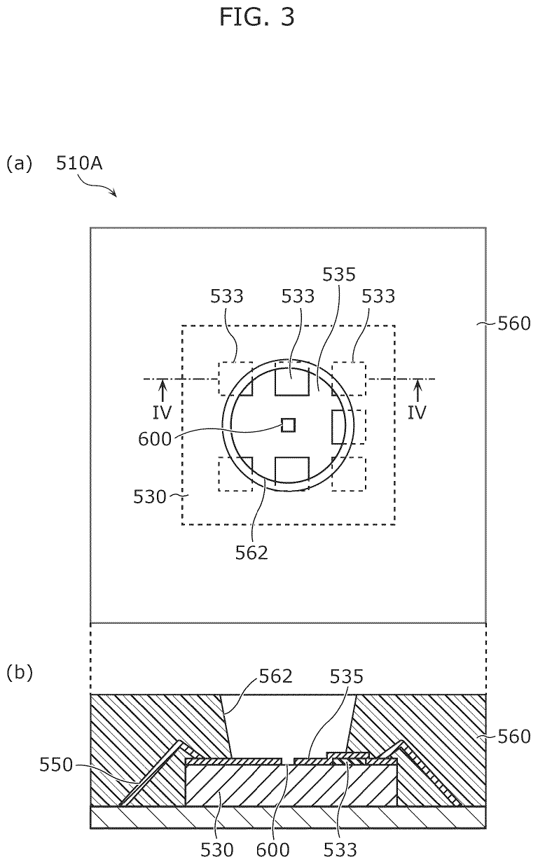

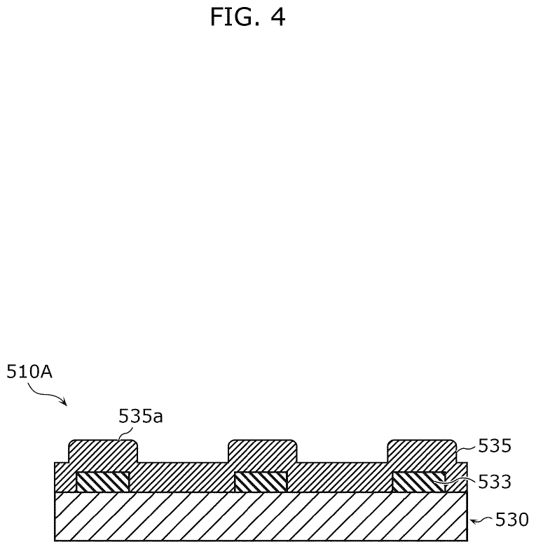

FIG. 3 is a diagram showing the positions of topmost wirings 533 and insulating film 535 of chip 530 of sensor package 510A according to Comparative Example 2. FIG. 4 is a diagram illustrating a cross section taken at the line IV-IV in FIG. 3.

In FIG. 3, (a) illustrates a plan view of sensor package 510A according to Comparative Example 2 while (b) illustrates a cross-sectional view of sensor package 510A. FIG. 4 illustrates topmost wirings 533 formed on the surface of chip 530 and insulating film 535 formed on the surface of chip 530 to cover topmost wirings 533.

As illustrated in (a) in FIG. 3, topmost wirings 533 are scattered on the surface of chip 530 and arranged like islands. For this reason, insulating film 535 is unevenly formed in such a manner to correspond to the positions of topmost wirings 533 (see FIG. 4). Therefore, problems indicated below may occur when resin is molded with protrusion 92 of mold 90 pressed against uneven surface 535a of insulating film 535.

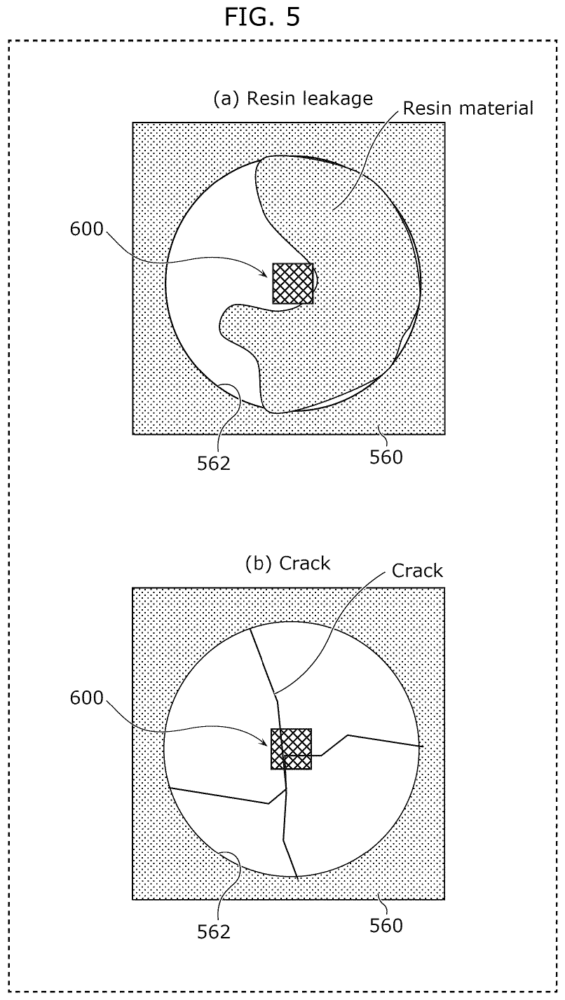

FIG. 5 is a diagram illustrating examples of a problem that occurs in sensor package 510A according to Comparative Example 2. In FIG. 5, (a) shows an example of an occurrence of resin leakage while (b) shows an example of an occurrence of cracks.

As described above, aperture 562 is formed by resin molding with protrusion 92 of mold 90 pressed against insulating film 535 of chip 530, but resin material intrudes through the gaps in the unevenness of insulating film 535 toward sensor unit 600 due to a strong pressure generated in mold 90. In this case, a problem is that the resin material comes into contact with sensor unit 600, as shown in (a) in FIG. 5, which reduces the detection accuracy of sensor unit 600.

To prevent the resin material from intruding toward sensor unit 600, it is conceivable to press protrusion 92 of mold 90 strongly against insulating film 535. However, a problem is that when protrusion 92 is pressed too hard, defects such as cracks may occur in chip 530, as shown in (b) in FIG. 5, making detection by sensor unit 600 impossible.

The sensor package or the like according to the present disclosure has a configuration described below to suppress deterioration in the quality of the sensor unit.

Hereinafter, embodiments of the present disclosure will be described in detail with reference to the drawings. The embodiments described below each show a specific example of the present disclosure. The numeric values, shapes, materials, elements, arrangement and connection of elements, steps, an order of steps, etc. indicated in the following embodiments are mere examples, and do not intend to limit the present disclosure. The figures are not necessarily precise illustrations. In the figures, elements that are essentially the same share like reference signs, and duplicate description is omitted or simplified.

In the present specification, terms indicating relationships between elements, such as “parallel”, and terms indicating the shape of elements, such as “rectangular”, as well as numerical ranges are expressions that do not express strict meaning only, but also mean substantially equivalent ranges, including differences of a few percent, for example.

In some of the figures, the X, Y, and Z axes meaning three mutually orthogonal directions are shown, and these axes and axial directions along the axes may be used for illustrative purposes when necessary. Each axis is provided to facilitate understanding and does not limit the direction and orientation in which the sensor package is used.

### Embodiment 1

### [Configuration of Sensor Package]

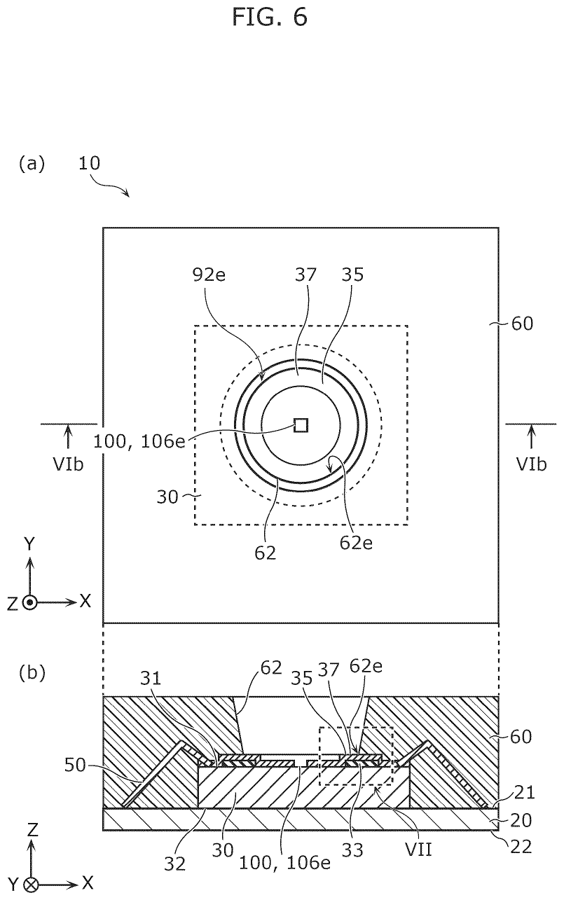

The configuration of a sensor package according to Embodiment 1 will be described with reference to FIG. 6 to FIG. 9.

FIG. 6 is a schematic diagram of sensor package 10 according to Embodiment 1. In FIG. 6, (a) illustrates a plan view of sensor package 10 and (b) illustrates a cross section taken at the line VIb-VIb in (a).

As illustrated in FIG. 6, sensor package 10 includes substrate 20, chip 30 mounted on substrate 20, and molded resin portion 60 provided on chip 30 and substrate 20.

Substrate 20 is, for example, a resin substrate or a ceramic substrate, and is plate-like in shape. Substrate 20 has surface 21 and back surface 22 facing away from surface 21. Surface 21 and back surface 22 are flat and are parallel to each other. Substrate 20 is rectangular in shape when viewed from a direction perpendicular to surface 21. Substrate 20 has multiple wirings and multiple pad electrodes, and the wirings are formed to electrically connect the external terminal on the back surface 22 side and the pad electrodes (not shown in the figure) on the surface 21 side.

Substrate 20 is not limited to a resin substrate or a ceramic substrate and may be a lead frame. Since the lead frame has less thickness variation, the thickness variation of the components (substrate, adhesive layer, and chip) sandwiched between the lower mold and protrusion 92 of the upper mold can be reduced. When substrate 20 is a lead frame, the die pads and pad sections may be separately arranged like islands, with each island located in a rectangular area. In the case of a lead frame substrate with multiple leads embedded with resin or the like, the shape of the lead frame substrate including resin and lead is rectangular.

Chip 30 is a semiconductor chip and is rectangular and plate-like in shape. The area of chip 30 is smaller than the area of substrate 20. Chip 30 includes surface 31 and back surface 32 facing away from surface 31. Chip 30 is disposed on substrate 20 so that back surface 32 faces surface 21 of substrate 20. In the cross-sectional view in (b) in FIG. 6, the illustration of a bonding layer (e.g., a die bonding agent or a die bonding film tape) is omitted. The same is true in subsequent cross-sectional views.

Chip 30 includes sensor unit 100 for detecting the environmental conditions (e.g., air quality) of the space in which sensor package 10 is placed. Sensor unit 100 is, for example, a hydrogen sensor that detects hydrogen. Sensor unit 100 may be an environment sensor that detects temperature, humidity, gas concentration, or airflow.

Exposed portion 106e of sensor unit 100 is provided on surface 31 of chip 30. Exposed portion 106e is provided in a position that is at the center of chip 30 and that corresponds to aperture 62 of molded resin portion 60. Exposed portion 106e of sensor unit 100 is positioned more inside than the side face of aperture 62.

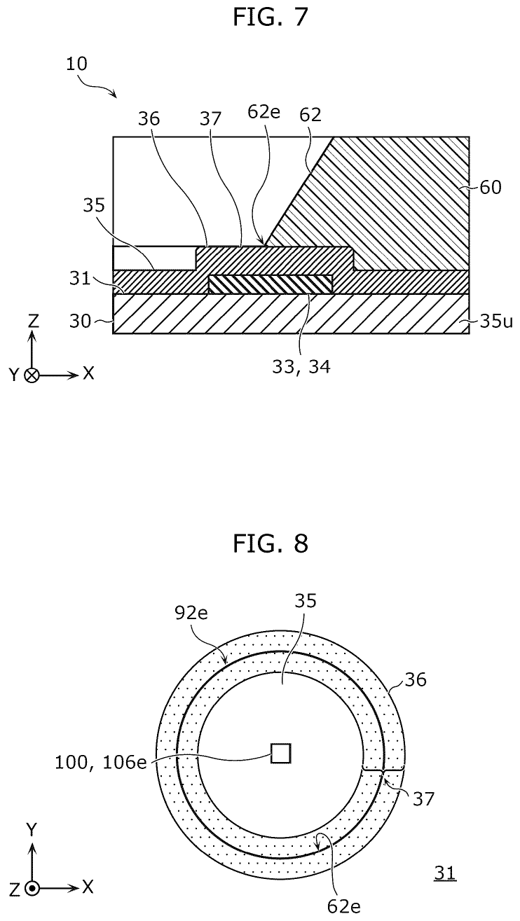

FIG. 7 is a diagram illustrating a cross section of chip 30 and molded resin portion 60 of sensor package 10. FIG. 7 shows an enlarged view of the part indicated by VII in FIG. 6.

Chip 30 includes multiple wirings and multiple insulating films. In this example, topmost wirings 33 are formed on lower insulating film 35u that is flattened. For example, some of topmost wirings 33 are each composed of dummy electrode 34 not electrically connected to sensor unit 100. Dummy electrode 34 has a predetermined thickness and is formed annularly outside of exposed portion 106e. Dummy electrode 34 in this example is annular in shape.

Topmost insulating film 35 is formed on lower insulating film 35u and topmost wirings 33. In this example, the insulating film formed on surface 31 of chip 30 is topmost insulating film 35. The material of topmost insulating film 35 is, for example, silicon nitride.

Insulating film 35 is provided on surface 31 of chip 30 to cover dummy electrode 34 having a predetermined thickness. For this reason, convex portion 36 is formed in insulating film 35 to correspond to a position in which dummy electrode 34 is provided. In other words, insulating film 35 includes convex portion 36 protruding in a direction perpendicular to surface 31 of chip 30, and convex portion 36 is formed by insulating film 35 covering over dummy electrode 34. This convex portion 36 is formed annularly to surround exposed portion 106e along the shape of dummy electrode 34.

Flat part 37, which is a flat area, is provided at the tip of annular convex portion 36. Flat part 37 is formed continuously flat without any steps in the circumferential direction that surrounds exposed portion 106e. For example, the width in the radial direction of flat part 37 is greater than or equal to 10 μm and the difference in height of irregularities is less than 1 μm.

FIG. 8 is the top view of flat part 37 of chip 30.

In FIG. 8, flat part 37 is indicated by hatched dots. Also, for reference, the position of edge 92e of protrusion 92 of mold 90 is indicated by an arrow. Hereinafter, the same is indicated in the top view of the flat part of chip 30.

As illustrated in FIG. 8, flat part 37 is positioned outside of exposed portion 106e on surface 31 of chip 30. Flat part 37, like convex portion 36, is formed annularly to surround exposed portion 106e. Flat part 37 in this example is annular in shape.

Chip 30 also includes multiple wirings drawn outwardly from sensor unit 100 and multiple pad electrodes (not shown in the figure) positioned at the edge portion outside the wirings. Pad electrodes of chip 30 are electrically connected to pad electrodes of substrate 20 via wires 50 (see FIG. 6). Part of chip 30, wires 50, and substrate 20 are covered by molded resin portion 60.

Molded resin portion 60 is formed by molding thermosetting resin. Molded resin portion 60 is provided on chip 30 and substrate 20 so as not to cover exposed portion 106e of sensor unit 100. In other words, molded resin portion 60 is formed to cover surface 31 of chip 30 excluding exposed portion 106e as well as surface 21 of substrate 20. FIG. 6 illustrates a structure in which the entire surface 21 of substrate 20 is covered with molded resin portion 60, but the structure is not limited to this example and part of substrate 20, such as the outer circumference of substrate 20, may not be covered by molded resin.

Molded resin portion 60 has aperture 62 positioned above exposed portion 106e. Aperture 62 is a ventilation hole leading from the outside to sensor unit 100 and penetrates molded resin portion 60 in the thickness direction of molded resin portion 60. In this example, a single aperture 62 is formed at the center of molded resin portion 60. Aperture 62 is formed in a tapered shape so that the area of the hole increases from the inside toward the outside. Aperture 62 is formed using protrusion 92 protruding from the inner side of mold 90 (see FIG. 2). When the shape of protrusion 92 is conical or cylindrical, for example, the shape of edge 62e of aperture 62 is circular. In this example, edge 62e of aperture 62 on the surface 31 side of chip 30 out of the edges of aperture 62 is positioned on convex portion 36 of insulating film 35.

In the present embodiment, when molded resin portion 60 is formed, edge 92e of protrusion 92 of mold 90 is pressed against flat part 37 of chip 30. This results in sensor package 10 after molding having a structure in which edge 62e of aperture 62 contacts flat part 37 of convex portion 36 and is formed along flat part 37 that is flat in the circumferential direction. Stated differently, edge 62e of aperture 62 is positioned in the same plane on the surface 31 side of chip 30.

According to the above-described structure, when molded resin portion 60 is formed, for example, it is unlikely that a gap is formed between protrusion 92 of mold 90 and flat part 37 of chip 30. For this reason, it is possible to inhibit resin material for forming molded resin portion 60 from intruding toward sensor unit 100. This can suppress deterioration in the quality of sensor unit 100.

According to the above-described structure, since the intrusion of the resin material can be inhibited, the pressing force of mold 90 against chip 30 can be reduced and the occurrence of cracks in chip 30 can be inhibited. Moreover, since the intrusion of the resin material can be inhibited, the sizes of aperture 62 and chip 30 can be reduced.

Although the above has illustrated an example in which the shape of flat part 37 of chip 30 is annular, the shape of flat part 37 is not limited to this example.

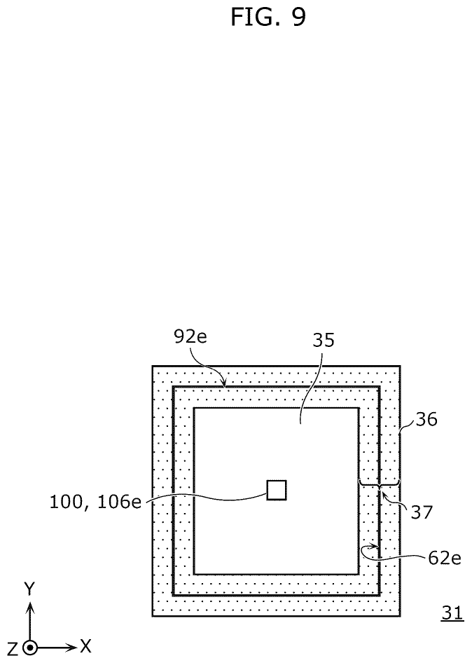

FIG. 9 is a diagram illustrating another example of flat part 37 of chip 30.

As illustrated in the figure, the shape of flat part 37 of chip 30 may be angular annular. The angles in this example are square or rectangular in shape. When the shape of flat part 37 is angular annular, the shape of convex portion 36 of insulating film 35 for forming flat part 37 as well as the shape of dummy electrode 34 for forming convex portion 36 may be also angular annular. The shape of protrusion 92 of mold 90 may be pyramidal or prismatic and the shape of edge 62e of aperture 62 may be angular.

### [Configuration of Sensor Unit]

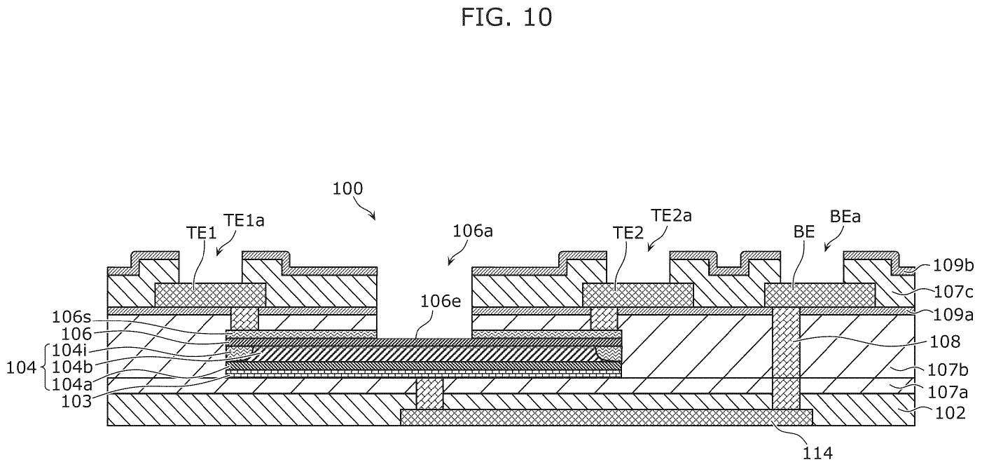

The configuration of sensor unit 100 of sensor package 10 will be described with reference to FIG. 10. Here, an example in which sensor unit 100 is a hydrogen sensor will be described.

FIG. 10 is a diagram illustrating a cross section of sensor unit 100 of sensor package 10.

Sensor unit 100 is a wide-range hydrogen sensor that detects low and high concentrations of hydrogen in a microscopic structure that can be fabricated in a semiconductor manufacturing process, and the main structures are: first electrode 103 and second electrode 106 arranged with their main surfaces facing each other; metal oxide layer 104 disposed in contact with the main surfaces of first electrode 103 and second electrode 106; insulating films 107a to 107c as well as 109a and 109b covering first electrode 103, second electrode 106, and metal oxide layer 104; first terminal TE1 and second terminal TE2 connected, via vias, to the other surface of second electrode 106 facing away from the main surface of second electrode 106; and third terminal BE connected, via a via, to the other surface of first electrode 103 facing away from the main surface of first electrode 103. Insulating film 107b includes opening 106 a that exposes the other surface of second electrode 106 between first terminal TE1 and second terminal TE2 in the plan view of second electrode 106, without being covered by insulating film 107b.

First electrode 103 is a surface electrode and has two faces. One face (i.e., the top surface in FIG. 10) of the two faces of first electrode 103 contacts metal oxide layer 104 and the other face (i.e., the bottom surface in FIG. 10) contacts insulating film 107a and via 108. First electrode 103 is rectangular in shape and has the same size as second electrode 106 when viewed from a direction perpendicular to the main surface. First electrode 103 may include material such as tungsten, nickel, tantalum, titanium, aluminum, tantalum nitride, and titanium nitride whose standard electrode potentials are lower than the standard electrode potential of a metal included in a metal oxide. The higher the value of the standard electrode potential, the more resistant to oxidation the value represents. First electrode 103 in FIG. 10 is formed by, for example, transition metal nitride such as tantalum nitride (TaN) or titanium nitride (TiN), or the laminations thereof.

Metal oxide layer 104 is sandwiched between two main surfaces, of first electrode 103 and second electrode 106, which face each other, includes a metal oxide serving as a gas-sensitive resistive film, and has a resistance value that varies reversibly according to the presence or absence of hydrogen content in the gas with which second electrode 106 comes into contact. Metal oxide layer 104 may have the property of changing resistance due to hydrogen. For example, metal oxide layer 104 includes an oxygen-deficient metal oxide. At least one of aluminum (Al) and transition metals such as tantalum (Ta), hafnium (Hf), titanium (Ti), zirconium (Zr), niobium (Nb), tungsten (W), nickel (Ni), and iron (Fe) may be selected for the base metal of metal oxide layer 104.

Since a transition metal can take multiple oxidization states, different resistive states are achievable by oxidation-reduction reactions. The “oxygen deficiency” of a metal oxide is the ratio of the amount of oxygen deficiency in the metal oxide to the amount of oxygen in an oxide of stoichiometric composition composed of the same elements as the metal oxide. The amount of oxygen deficiency is a value obtained by subtracting the amount of oxygen in the metal oxide from the amount of oxygen in the metal oxide in the stoichiometric composition. If there is a plurality of metal oxides in the stoichiometric composition composed of the same elements as the metal oxide, the oxygen deficiency of the metal oxide is defined based on one metal oxide having the highest resistance value among the metal oxides in the stoichiometric composition. The metal oxide in the stoichiometric composition has a more stable and higher resistance value compared to a metal oxide in other composition.

When the base metal of metal oxide layer 104 is tantalum (Ta), for example, since the oxide in the stoichiometric composition according to the above definition is Ta₂O₅, the oxide can be expressed as TaO_{2.5}. The oxygen deficiency of TaO_{2.5} is 0% and the oxygen deficiency of TaO_{1.5} is (2.5−1.5)/2.5=40%. Moreover, metal oxides with excess oxygen have negative oxygen deficiency. In the present disclosure, unless otherwise noted, oxygen deficiency can be positive, zero, or negative. An oxide with low oxygen deficiency is closer to the oxide in the stoichiometric composition and therefore has a higher resistivity, while an oxide with high oxygen deficiency is closer to a metal included in the oxide and therefore has a lower resistivity.

Metal oxide layer 104 illustrated in FIG. 10 includes: first layer 104a that contacts first electrode 103; second layer 104b that contacts first layer 104a and second electrode 106; and isolation layer 104i. The oxygen deficiency of second layer 104b is less than that of first layer 104a. First layer 104a is, for example, TaOₓ. Second layer 104b is Ta₂O₅ with lower oxygen deficiency than first layer 104a. Metal oxide layer 104 also includes isolation layer 104i at the outer circumference in the plan view of first electrode 103.

The expression “in the plan view” used herein means that sensor unit 100 according to the present disclosure is viewed from a viewpoint in the stacking direction of sensor unit 100 in FIG. 10, stated differently, sensor unit 100 is viewed from a viewpoint in the normal direction of any plane of, for instance, surface first electrode 103 or surface second electrode 106, and for example, the top surface of sensor unit 100 is viewed from a direction perpendicular to the main surface of sensor unit 100.

The resistive state of such metal oxide layer 104 depends on hydrogen-containing gas in contact with second electrode 106, and the resistance value decreases as the amount of the hydrogen-containing gas increases. Specifically, when hydrogen-containing gas is present in air to be detected, hydrogen atoms are dissociated from hydrogen-containing gas in second electrode 106. The dissociated hydrogen atoms intrude into metal oxide layer 104 and form impurity levels. In particular, the dissociated hydrogen atoms are concentrated near the interface with second electrode 106, apparently making the thickness of second layer 104b thinner. As a result, the resistance value of metal oxide layer 104 decreases.

Second electrode 106 is a hydrogen dissociative surface electrode and has two faces. One face (i.e., the bottom surface in FIG. 10) of the two faces of second electrode 106 contacts metal oxide layer 104 and the other face (i.e., the top surface in FIG. 10) contacts metal layer 106s and open air. Second electrode 106 includes, inside of opening 106a, exposed portion 106e that is exposed to open air. Second electrode 106 includes material, which has a catalytic action that dissociates hydrogen atoms from gas molecule including hydrogen atoms, such as precious metals like platinum (Pt), iridium (Ir), and palladium (Pd), or nickel (Ni), or alloys containing at least one of these. Second electrode 106 in FIG. 10 is assumed to be platinum (Pt). Two terminals, i.e., first terminal TE1 and second terminal TE2, are connected to second electrode 106.

First terminal TE1 is connected to second electrode 106 via via 108.

Second terminal TE2 is connected to second electrode 106 via via 108. First terminal TE1 and second terminal TE2 are connected, via openings TE1a and TE2a, to an external detection circuit that drives sensor unit 100.

First terminal TE1 and second terminal TE2 are arranged in positions that sandwich exposed portion 106e in the plan view of second electrode 106. This arrangement allows exposed portion 106e of second electrode 106 to be energized by the application of a predetermined voltage between first terminal TE1 and second terminal TE2, i.e., current flows through exposed portion 106e. This energization of exposed portion 106e of second electrode 106 is considered to activate the hydrogen dissociation action of exposed portion 106e. The predetermined voltage may be a voltage having mutually opposite polarities.

Sensor unit 100 changes the resistance between first terminal TE1 and second terminal TE2 when gas molecules containing hydrogen atoms touch exposed portion 106e while exposed portion 106e is energized. As a result of the detection circuit detecting this change in the resistance (this detection is also referred to as “lateral mode”), gas molecule containing hydrogen atoms with low concentration is detected.

Third terminal BE is connected to first electrode 103 via opening BEa, via 108, wiring 114, and via 108. Third terminal BE is connected, via opening BEa, to an external detection circuit that drives sensor unit 100. Sensor unit 100 changes the resistance between first electrode 103 and second electrode 106 when gas molecules containing hydrogen atoms touch exposed portion 106e while exposed portion 106e is energized. Stated differently, sensor unit 100 changes the resistance between third terminal BE and at least one of first terminal TE1 or second terminal TE2 when gas molecules containing hydrogen atoms touch exposed portion 106e while exposed portion 106e is energized. As a result of the detection circuit detecting this change in the resistance (this detection is also referred to as “vertical mode”), gas molecule containing hydrogen atoms with high concentration is detected.

Insulating film 102, insulating films 107a to 107c, and insulating films 109a and 109b, which cover the major part of sensor unit 100, are formed by, for instance, a silicon oxide film, and a silicon nitride film.

Moreover, metal layer 106s is formed on the top surface of second electrode 106 excluding opening 106a. Metal layer 106s includes, for example, TiAlN as material, and is formed as an etching stopper for the formation of via 108, but this is not essential.

The laminate of first electrode 103, metal oxide layer 104, and second electrode 106 is a device that can be used as a storage element for resistance change memory (ReRAM). The resistance change memory uses two of the possible states of metal oxide layer 104, a high resistive state and a low resistive state, to create a digital storage element. Sensor unit 100 according to the present disclosure uses the high resistive state among the possible states of metal oxide layer 104.

In FIG. 10, metal oxide layer 104 is shown as having a two-layer structure including first layer 104a that includes TaOₓ as material and second layer 104b that includes Ta₂O₅ with low oxygen deficiency as material, but metal oxide layer 104 may include one layer including Ta₂O₅ or TaOₓ with low oxygen deficiency as material.

FIG. 10 illustrates a schematic diagram in which openings TE1a, TE2a, and BEa are arranged near exposed portion 106e, but the arrangement of the openings is not limited to this example. For example, openings TE1a, TE2a, and BEa may be arranged distant from exposed portion 106e and outside of edge 62e of aperture 62. Openings TE1a, TE2a, and BEa may be arranged at the periphery of chip 30 as the openings of the pad electrode portions.

### [Sensor Package Manufacturing Method]

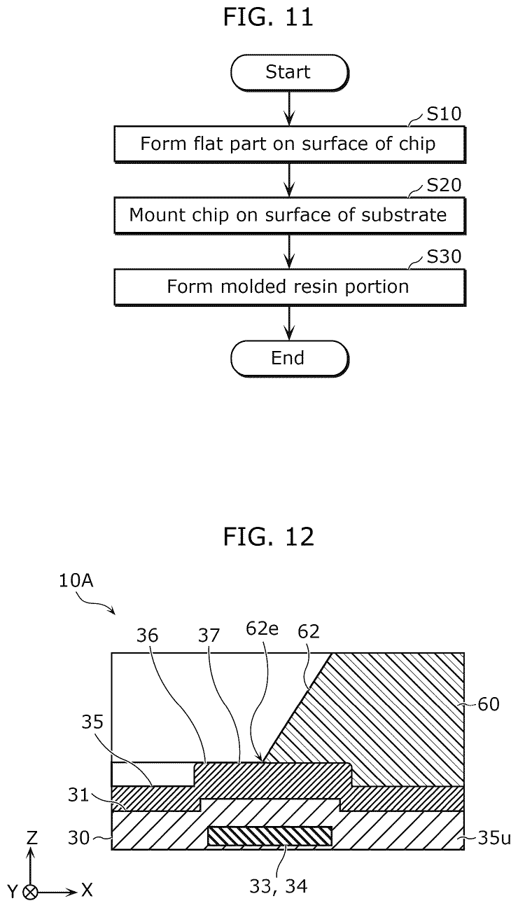

A method of manufacturing sensor package 10 according to Embodiment 1 will be described with reference to FIG. 11.

FIG. 11 is a flowchart illustrating the manufacturing method of sensor package 10.

The manufacturing method of sensor package 10 illustrated in FIG. 11 includes step S10 of forming flat part 37 on surface 31 of chip 30, step S20 of mounting chip 30 on surface 21 of substrate 20, and step S30 of forming molded resin portion 60.

First, in step S10, flat part 37 is provided outside of an area in which exposed portion 106e of sensor unit 100 is formed. Flat part 37 may be provided in chip 30 before or after exposed portion 106e is formed.

Subsequently in step S20, chip 30 is mounted on substrate 20 by wire bonding. With this, chip 30 and substrate 20 are electrically connected to each other via wires 50.

Subsequently in step S30, protrusion 92 of mold 90 is arranged above exposed portion 106e of sensor unit 100 of chip 30, and molded resin portion 60 is formed to cover surface 31 of chip 30 excluding exposed portion 106e, as well as surface 21 of substrate 20. In this example, mold 90 is arranged so that edge 92e of protrusion 92 is positioned on flat part 37, to mold resin. When the resin is molded, a film-like resin sheet may be placed along the inner face of mold 90 to form molded resin portion 60.

Thus, when molded resin portion 60 is formed, by arranging edge 92e of protrusion 92 of mold 90 on flat part 37, it is possible to inhibit a gap from being formed between protrusion 92 and flat part 37. For this reason, it is possible to inhibit resin material for forming molded resin portion 60 from intruding toward sensor unit 100. This can suppress deterioration in the quality of sensor unit 100.

### Variation 1 of Embodiment 1

Sensor package 10A according to Variation 1 of Embodiment 1 will be described. In Variation 1, an example in which another insulating film is formed between dummy electrode 34 and convex portion 36 will be described.

Sensor package 10A according to Variation 1 includes substrate 20, chip 30 mounted on substrate 20, and molded resin portion 60 provided on chip 30 and substrate 20. The configurations of substrate 20 and molded resin portion 60 are the same as those described in Embodiment 1.

FIG. 12 is a diagram illustrating a cross section of chip 30 and molded resin portion 60 of sensor package 10A according to Variation 1.

Chip 30 has multiple wirings and multiple insulating films. Dummy electrode 34 has a predetermined thickness and is formed annularly outside of exposed portion 106e.

In Variation 1, lower insulating film 35u is formed over dummy electrode 34, and topmost insulating film 35 is formed on lower insulating film 35u.

Lower insulating film 35u is provided in chip 30 to cover dummy electrode 34 having the predetermined thickness. For this reason, lower insulating film 35u is provided with a convex portion formed in such a manner to correspond to a position in which dummy electrode 34 is provided. Topmost insulating film 35 is provided on surface 31 of chip 30 to cover the convex portion of lower insulating film 35u. For this reason, with convex portion 36 formed in topmost insulating film 35 in such a manner to correspond to a position in which dummy electrode 34 and the convex portion of lower insulating film 35u are provided.

In other words, insulating film 35 includes convex portion 36 protruding in a direction perpendicular to surface 31 of chip 30, and convex portion 36 is formed by insulating film 35 covering over dummy electrode 34. This convex portion 36 is formed annularly to surround exposed portion 106e along the shape of dummy electrode 34.

In sensor package 10A according to Variation 1, edge 62e of aperture 62 contacts flat part 37 of convex portion 36 and is formed along flat part 37 that is flat in the circumferential direction. In other words, edge 62e of aperture 62 is positioned in the same plane on the surface 31 side of chip 30. According to this structure, the same advantageous effects as those described in Embodiment 1 can be achieved.

### Variation 2 of Embodiment 1

Sensor package 10B according to Variation 2 of Embodiment 1 will be described. In Variation 2, an example in which chip 30 includes two or more annular flat parts 37 will be described.

Sensor package 10B according to Variation 2 includes substrate 20, chip 30 mounted on substrate 20, and molded resin portion 60 provided on chip 30 and substrate 20. The configuration of substrate 20 is the same as that described in Embodiment 1.

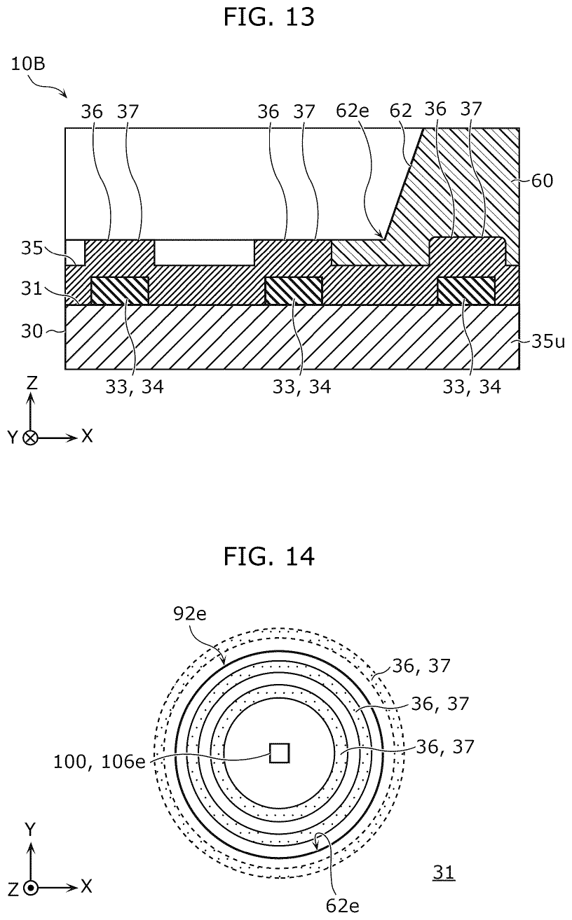

FIG. 13 is a diagram illustrating a cross section of chip 30 and molded resin portion 60 of sensor package 10B according to Variation 2.

Chip 30 includes multiple wirings and multiple insulating films. Topmost wirings 33 include a plurality of dummy electrodes 34. Dummy electrode 34 has a predetermined thickness and is formed annularly outside of exposed portion 106e. The plurality of dummy electrodes 34 include two or more annular dummy electrodes 34.

Insulating film 35 is provided on surface 31 of chip 30 to cover the plurality of dummy electrodes 34. For this reason, insulating film 35 is provided with a plurality of convex portions 36 formed in such a manner to correspond to positions in which the plurality of dummy electrodes 34 are provided. In other words, insulating film 35 includes the plurality of convex portions 36 protruding in a direction perpendicular to surface 31 of chip 30, and the plurality of convex portions 36 are formed by insulating film 35 covering over dummy electrodes 34. These convex portions 36 are formed as two or more rings to surround exposed portion 106e along the shapes of dummy electrodes 34. Annular concave portions (opening grooves) are formed between the plurality of convex portions 36.

Flat part 37, which is a flat area, is provided at the tip of annular convex portion 36. A plurality of flat parts 37 formed corresponding to the plurality of convex portions 36 are formed in the same plane. A difference in the height of flat parts 37 formed at the plurality of convex portions 36 is, for example, less than 1 μm. The above illustrates an example in which convex portion 36 is formed by covering only dummy electrode 34 with insulating film 35, but the formation of convex portion 36 is not limited to this example. For example, convex portion 36 may be formed by covering, with insulating film 35, wiring in which a dummy electrode and wiring for electrical connection are combined.

FIG. 14 is the top view of flat parts 37 of chip 30 according to Variation 2.

The plurality of flat parts 37 include two or more annular flat parts 37. Each flat part 37 is positioned outside of exposed portion 106e on surface 31 of chip 30. Each flat part 37, like each convex portion 36, is formed annularly to surround exposed portion 106e.

Molded resin portion 60 has aperture 62 positioned above exposed portion 106e. Aperture 62 is a ventilation hole leading from the outside to sensor unit 100 and penetrates molded resin portion 60 in the thickness direction of molded resin portion 60. Aperture 62 is formed using protrusion 92 protruding from the inner side of mold 90. When the shape of protrusion 92 is conical or cylindrical, for example, the shape of edge 62e of aperture 62 is circular.

In the present variation, when molded resin portion 60 is formed, the bottom of protrusion 92 of mold 90 is pressed against inner flat part 37 excluding outermost flat part 37. This results in sensor package 10B after molding having a structure in which edge 62e of aperture 62 is positioned between the outermost convex portion 36 and the second outermost convex portion 36. Edge 62e of aperture 62 is formed along flat part 37 of the outermost convex portion 36 and flat part 37 of the second outermost convex portion 36. The height of edge 62e is the same as the height of these flat parts 37.

According to the above-described structure, when molded resin portion 60 is formed, for example, it is unlikely that a gap is formed between protrusion 92 of mold 90 and flat parts 37 of chip 30. For this reason, it is possible to inhibit resin material for forming molded resin portion 60 from intruding toward sensor unit 100. Specifically, since chip 30 includes one or more annular flat parts 37, when molded resin portion 60 is formed, the bottom of protrusion 92 contacts the one or more flat parts 37 and leaking of the resin material toward sensor unit 100 can be inhibited. This can suppress deterioration in the quality of sensor unit 100. Furthermore, with two or more circular patterns, it is possible to trap resin that has intruded into annular concave portions (opening grooves) between the plurality of convex portions 36 even when the resin intrudes beyond the outer circular pattern. For this reason, it is possible to reduce the risk that sensor unit 100 is covered with resin even beyond the innermost circular pattern.

Although the above has illustrated an example in which the shape of flat part 37 of chip 30 is annular, the shape of flat part 37 is not limited to this example.

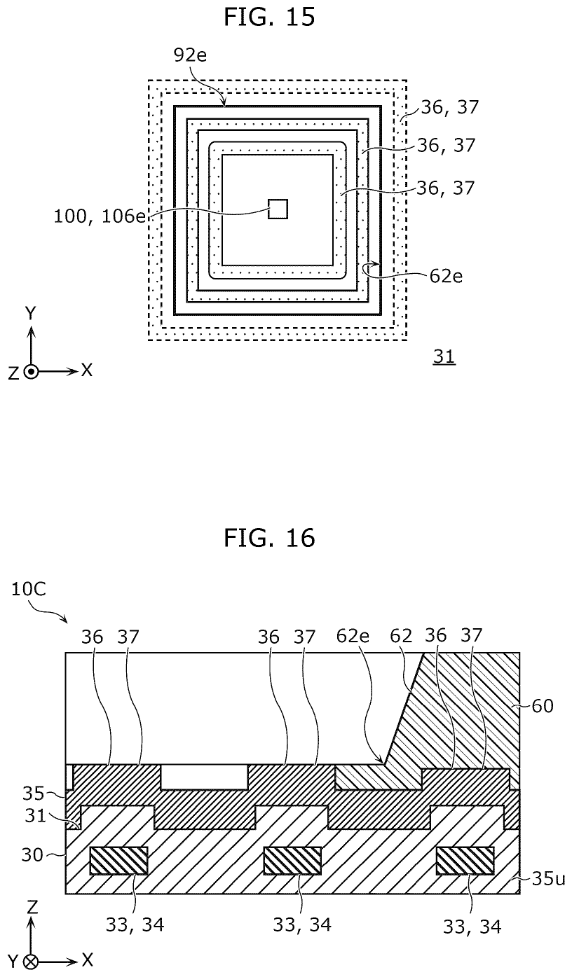

FIG. 15 is a diagram illustrating another example of flat parts 37 of chip 30.

As illustrated in the figure, the shape of flat parts 37 of chip 30 may be angular annular. The angles in this example are square or rectangular in shape. When the shape of flat parts 37 is angular annular, the shape of convex portions 36 of insulating film 35 for forming flat parts 37 as well as the shape of dummy electrodes 34 for forming convex portions 36 may be also angular annular. The shape of protrusion 92 of mold 90 may be pyramidal or prismatic and the shape of edge 62e of aperture 62 may be angular.

### Variation 3 of Embodiment 1

Sensor package 10C according to Variation 3 of Embodiment 1 will be described. In Variation 3, an example in which another insulating film is formed between dummy electrode 34 and convex portion 36 in the sensor package according to Variation 2 will be described.

Sensor package 10C according to Variation 3 includes substrate 20, chip 30 mounted on substrate 20, and molded resin portion 60 provided on chip 30 and substrate 20. The configurations of substrate 20 and molded resin portion 60 are the same as those described in Variation 2.

FIG. 16 is a diagram illustrating a cross section of chip 30 and molded resin portion 60 of sensor package 10C according to Variation 3.

Chip 30 includes multiple wirings and multiple insulating films. Dummy electrode 34 has a predetermined thickness and is formed annularly outside of exposed portion 106e.

In Variation 3, lower insulating film 35u is formed over dummy electrodes 34, and topmost insulating film 35 is formed on lower insulating film 35u.

Lower insulating film 35u is provided in chip 30 to cover dummy electrodes 34 each having a predetermined thickness. For this reason, convex portions are formed in lower insulating film 35u to correspond to positions in which dummy electrodes 34 are provided. Topmost insulating film 35 is provided on surface 31 of chip 30 to cover the convex portions of lower insulating film 35u. For this reason, convex portions 36 are formed in topmost insulating film 35 in such a manner to correspond to the positions in which dummy electrodes 34 and the convex portions of lower insulating film 35u are provided.

In other words, insulating film 35 has convex portions 36 protruding in a direction perpendicular to surface 31 of chip 30, and convex portions 36 are formed by insulating film 35 covering over dummy electrodes 34. These convex portions 36 are formed annularly to surround exposed portion 106e along the shapes of dummy electrodes 34.

In sensor package 10C according to Variation 3, resin is molded with the bottom of protrusion 92 of mold 90 pressed against inner flat part 37. Edge 62e of aperture 62 is positioned in the same plane on the surface 31 side of chip 30. According to this structure, the same advantageous effects as those described in Embodiment 1 can be achieved.

In Variation 3, since chip 30 includes one or more annular flat parts 37, when molded resin portion 60 is formed, the bottom of protrusion 92 contacts flat part 37 and leaking of the resin material toward sensor unit 100 can be inhibited. This can suppress deterioration in the quality of sensor unit 100. Furthermore, with two or more circular patterns, it is possible to trap resin that has intruded into the annular concave portions (opening grooves) between the plurality of convex portions 36 even when the resin intrudes beyond the outer circular pattern. For this reason, it is possible to reduce the risk that sensor unit 100 is covered with resin even beyond the innermost circular pattern.

### Variation 4 of Embodiment 1

Sensor package 10D according to Variation 4 of Embodiment 1 will be described. In Variation 4, an example in which chip 30 has two or more annular flat parts 37 and opening grooves 38 are provided in insulating film 35 of chip 30 will be described.

Sensor package 10D according to Variation 4 includes substrate 20, chip 30 mounted on substrate 20, and molded resin portion 60 provided on chip 30 and substrate 20. The configuration of substrate 20 is the same as that described in Variation 3.

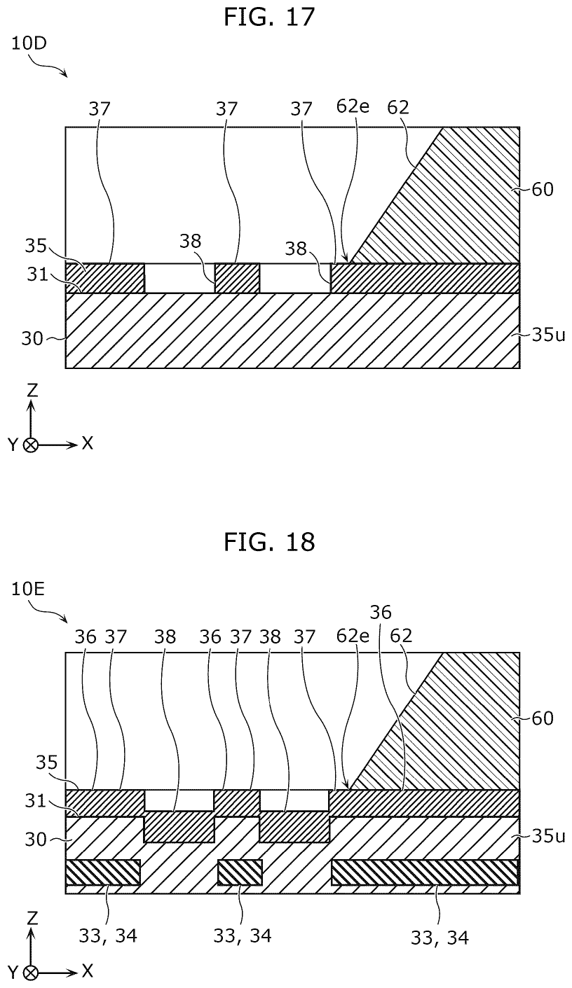

FIG. 17 is a diagram illustrating a cross section of chip 30 and molded resin portion 60 of sensor package 10D according to Variation 4.

Chip 30 includes multiple insulating films. Topmost insulating film 35 is formed on lower insulating film 35u that is flattened. Topmost insulating film 35 includes flat parts 37 that are flat areas and opening grooves 38 that are recessed with respect to flat parts 37. Each of opening grooves 38 penetrates topmost insulating film 35 in the thickness direction of topmost insulating film 35 so that lower insulating film 35u is exposed. Opening grooves 38 include two or more annular opening grooves 38. Annular opening grooves 38 are formed in insulating film 35, which divides flat part 37 into inner and outer sections. Flat parts 37 resulting from the division are formed annularly to surround exposed portion 106e along the shapes of opening grooves 38. Opening groove 38 in this example is annular in shape, and flat parts 37 include two or more circular flat parts 37. Flat parts 37 are formed in the same plane, and the difference in height of flat parts 37 is, for example, less than 1 μm. Although the above illustrates an example in which opening grooves 38 penetrate insulating film 35, opening grooves 38 are not limited to this example. For example, opening grooves 38 may be annular recesses (annular grooves) that leave the bottom of insulating film 35.

Molded resin portion 60 has aperture 62 positioned above exposed portion 106e. Aperture 62 is a ventilation hole leading from the outside to sensor unit 100 and penetrates molded resin portion 60 in the thickness direction of molded resin portion 60. Aperture 62 is formed using protrusion 92 protruding from the inner side of mold 90. When the shape of protrusion 92 is conical or cylindrical, for example, the shape of edge 62e of aperture 62 is circular.

In the present variation, when molded resin portion 60 is formed, edge 92e of protrusion 92 of mold 90 is pressed against the outermost flat part 37 of flat parts 37. This results in sensor package 10D after molding having a structure in which edge 62e of aperture 62 contacts the outermost flat part 37 and is formed along the outermost flat part 37. Stated differently, edge 62e of aperture 62 is positioned in the same plane on the surface 31 side of chip 30.

According to the above-described structure, when molded resin portion 60 is formed, for example, it is unlikely that a gap is formed between protrusion 92 of mold 90 and flat part 37 of chip 30. Specifically, since chip 30 includes one or more annular flat parts 37, when molded resin portion 60 is formed, the bottom of protrusion 92 contacts flat part 37 and leaking of resin material toward sensor unit 100 can be inhibited. This can suppress deterioration in the quality of sensor unit 100. Furthermore, with two or more circular patterns, it is possible to trap resin that has intruded into the annular concave portions (opening grooves) between the plurality of convex portions 36 even when the resin intrudes beyond the outer circular pattern. For this reason, it is possible to reduce the risk that sensor unit 100 is covered with resin even beyond the innermost circular pattern.

Although the above has illustrated an example in which the shape of flat parts 37 of chip 30 is circular, the shape of flat parts 37 may be annular or angular annular. The outermost flat part 37 that edge 92e of protrusion 92 of mold 90 contacts does not necessarily have to be circular and may be partially concave.

### Variation 5 of Embodiment 1

Sensor package 10E according to Variation 5 of Embodiment 1 will be described. In Variation 5, an example in which another insulating film is formed between dummy electrode 34 and convex portion 36 in the sensor package according to Variation 4 will be described.

Sensor package 10E according to Variation 5 includes substrate 20, chip 30 mounted on substrate 20, and molded resin portion 60 provided on chip 30 and substrate 20. The configurations of substrate 20 and molded resin portion 60 are the same as those described in Variation 4.

FIG. 18 is a diagram illustrating a cross section of chip 30 and molded resin portion 60 of sensor package 10E according to Variation 5.

Chip 30 includes multiple wirings and multiple insulating films. Dummy electrode 34 has a predetermined thickness and is formed annularly outside of exposed portion 106e.

In Variation 5, lower insulating film 35u is formed over dummy electrodes 34, and topmost insulating film 35 is formed on lower insulating film 35u.

Lower insulating film 35u is provided in chip 30 to cover dummy electrodes 34 each having a predetermined thickness. For this reason, convex portions are formed in lower insulating film 35u to correspond to positions in which dummy electrodes 34 are provided. Topmost insulating film 35 is provided on surface 31 of chip 30 to cover the convex portions of lower insulating film 35u. For this reason, convex portions 36 are formed in topmost insulating film 35 in such a manner to correspond to positions in which dummy electrodes 34 and the convex portions of lower insulating film 35u are provided.

In other words, insulating film 35 includes convex portions 36 protruding in a direction perpendicular to surface 31 of chip 30, and convex portions 36 are formed by insulating film 35 covering over dummy electrodes 34. These convex portions 36 are formed annularly to surround exposed portion 106e along the shapes of dummy electrodes 34. The tips of convex portions 36 are flat parts 37.

Insulating film 35 according to Variation 5 includes: flat parts 37 at the tips of convex portions 36; and a plurality of opening grooves 38 that are recessed with respect to flat parts 37. The plurality of opening grooves 38 include two or more annular opening grooves 38. Annular opening grooves 38 are formed in insulating film 35, which divides flat part 37 into inner and outer sections. Flat parts 37 resulting from the division are formed annularly to surround exposed portion 106e along the shapes of opening grooves 38. The shape of opening grooves 38 in this example is annular, and flat parts 37 include two or more annular flat parts 37. Flat parts 37 are formed in the same plane and the difference in height of flat parts 37 is, for example, less than 1 μm.

Molded resin portion 60 has aperture 62 positioned above exposed portion 106e. Aperture 62 is a ventilation hole leading from the outside to sensor unit 100 and penetrates molded resin portion 60 in the thickness direction of molded resin portion 60. Aperture 62 is formed using protrusion 92 protruding from the inner side of mold 90. When the shape of protrusion 92 is conical or cylindrical, for example, the shape of edge 62e of aperture 62 is circular.

In the present variation, when molded resin portion 60 is formed, edge 92e of protrusion 92 of mold 90 is pressed against the outermost flat part 37 of flat parts 37. This results in sensor package 10E after molding having a structure in which edge 62e of aperture 62 contacts the outermost flat part 37 and is formed along the outermost flat part 37. Stated differently, edge 62e of aperture 62 is positioned in the same plane on the surface 31 side of chip 30.

According to the above-described structure, when molded resin portion 60 is formed, for example, it is unlikely that a gap is formed between protrusion 92 of mold 90 and flat parts 37 of chip 30. For this reason, it is possible to inhibit resin material for forming molded resin portion 60 from intruding toward sensor unit 100. Since chip 30 includes two or more annular flat parts 37, leaking of the resin material toward sensor unit 100 can be inhibited. This can suppress deterioration in the quality of sensor unit 100.

Although the above has illustrated an example in which the shape of flat parts 37 of chip 30 is circular, the shape of flat parts 37 may be annular or angular annular. The outermost flat part 37 that edge 92e of protrusion 92 of mold 90 contacts does not necessarily have to be circular and may be partially concave. A pattern below the outermost flat part 37 that edge 92e of protrusion 92 contacts does not necessarily have to be circular and may be, for instance, wiring for electrical connection or an island-like dummy pattern.

Although the above has illustrated an example of forming convex portion 36 by covering only dummy electrode 34 with insulating film 35, the formation of convex portion 36 is not limited to this example. For example, convex portion 36 may be formed by covering, with insulating film 35, wiring in which a dummy electrode and wiring for electrical connection are combined.

### Variation 6 of Embodiment 1

Sensor package 10F according to Variation 6 of Embodiment 1 will be described. In Variation 6, an example in which protective film 45 is formed on insulating film 35 will be described.

Sensor package 10F according to Variation 6 includes substrate 20, chip 30 mounted on substrate 20, and molded resin portion 60 provided on chip 30 and substrate 20. The configuration of substrate 20 is the same as that described in Embodiment 1.

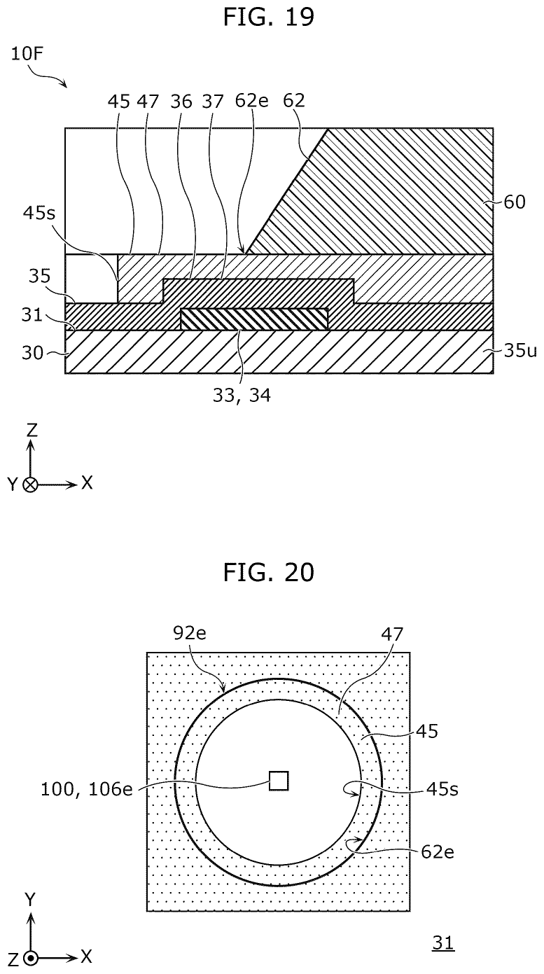

FIG. 19 is a diagram illustrating a cross section of chip 30 and molded resin portion 60 of sensor package 10F according to Variation 6.

In Variation 6, protective film 45 is formed to cover convex portion 36 of insulating film 35. Protective film 45 is formed by, for example, a polyimide resin or a fluorine resin. For example, protective film 45 is provided on surface 31 of chip 30 to cover convex portion 36 of insulating film 35 and not to cover exposed portion 106e of sensor unit 100. Inner side face 45s of protective film 45 is positioned in proximity to exposed portion 106e relative to convex portion 36 and is positioned in proximity to edge 62e of aperture 62 relative to exposed portion 106e.

Protective film 45 on convex portion 36 is provided with flat part 47 that is a flat area. Flat part 47 is formed continuously flat without any steps in the circumferential direction that surrounds exposed portion 106e. The difference in height of irregularities of flat part 47 is, for example, less than 1 μm. Since the flat area of protective film 45 needs to be formed corresponding to an area that protrusion 92 of mold 90 touches, an area that is outwardly little distant from edge 92e of protrusion 92 does not necessarily need to be flat and a protective film itself need not be formed.

FIG. 20 is the top view of flat part 47 of chip 30 according to Variation 6.

In FIG. 20, flat part 47 is indicated by hatched dots. The same is true in FIG. 22 to be described below.

Flat part 47 is positioned outside of exposed portion 106e on surface 31 of chip 30. Like convex portion 36, flat part 47 is formed to surround exposed portion 106e. The inner circumference (inner side face 45s) of flat part 47 in this example is circular in shape.

Molded resin portion 60 has aperture 62 positioned above exposed portion 106e. Aperture 62 is a ventilation hole leading from the outside to sensor unit 100 and penetrates molded resin portion 60 in the thickness direction of molded resin portion 60. Aperture 62 is formed using protrusion 92 protruding from the inner side of mold 90. When the shape of protrusion 92 is conical or cylindrical, for example, the shape of edge 62e of aperture 62 is circular. In this example, edge 62e of aperture 62 is positioned on protective film 45 above convex portion 36.

In the present variation, when molded resin portion 60 is formed, edge 92e of protrusion 92 of mold 90 is pressed against protective film 45. This results in sensor package 10F after molding having a structure in which edge 62e of aperture 62 contacts flat part 47 of protective film 45 and is formed along flat part 47 of protective film 45. Stated differently, edge 62e of aperture 62 is positioned in the same plane on the surface 31 side of chip 30.

According to the above-described structure, when molded resin portion 60 is formed, for example, it is unlikely that a gap is formed between protrusion 92 of mold 90 and flat part 47 of chip 30. For this reason, it is possible to inhibit resin material for forming molded resin portion 60 from intruding toward sensor unit 100. This can suppress deterioration in the quality of sensor unit 100.

According to the above-described structure, even when resin material for forming molded resin portion 60 intrudes between protective film 45 and protrusion 92, for example, surface tension guides the resin material circumferentially along the inner circumferential edge of protrusion 92, and the intrusion of the resin material can be stopped at the inner circumferential edge of protective film 45. This can suppress deterioration in the quality of sensor unit 100.

Although the above has illustrated an example in which the inner circumference of flat part 47 of chip 30 is circular in shape, the shape of flat part 47 is not limited to this example. The inner circumference of flat part 47 of chip 30 may be angular.

Although the above has illustrated an example in which convex portion 36 is formed due to annular dummy electrode 34 below flat part 47 of protective film 45, the formation of convex portion 36 is not limited to this example. For example, dummy electrode 34 for forming convex portion 36 may be absent or may be a non-circular pattern (e.g., wiring for electrical connection or an island-like dummy pattern).

### Variation 7 of Embodiment 1

Sensor package 10G according to Variation 7 of Embodiment 1 will be described. In Variation 7, an example in which two or more annular protective films 45 are formed will be described.

Sensor package 10G according to Variation 7 includes substrate 20, chip 30 mounted on substrate 20, and molded resin portion 60 provided on chip 30 and substrate 20. The configurations of substrate 20 and molded resin portion 60 are the same as those described in Variation 6.

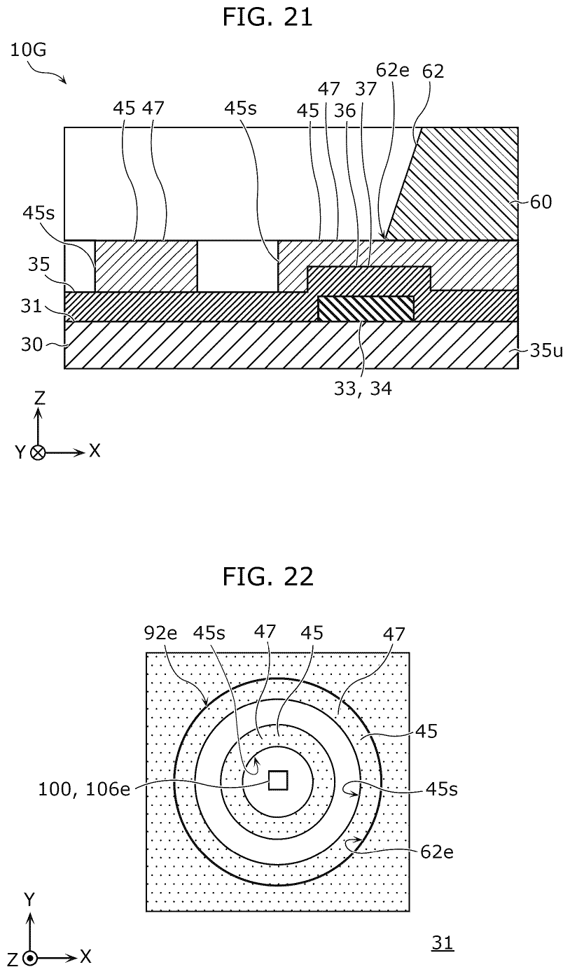

FIG. 21 is a diagram illustrating a cross section of chip 30 and molded resin portion 60 of sensor package 10G according to Variation 7.

In Variation 7, two or more annular protective films 45 are formed. Part of protective films 45 is formed to cover convex portion 36 of insulating film 35, and part of the remaining of protective films 45 is formed at a distance inward from protective film 45 covering convex portion 36.

These protective films 45 are each provided with flat part 47 that is a flat area. Flat part 47 is formed continuously flat without any steps in the circumferential direction that surrounds exposed portion 106e.

FIG. 22 is the top view of flat parts 47 of chip 30 according to Variation 7.

Flat parts 47 are positioned outside of exposed portion 106e on surface 31 of chip 30. Flat parts 47 of protective films 45 are formed to surround exposed portion 106e. The inner circumference (inner side face 45s) of each flat part 47 in this example is circular in shape.

Molded resin portion 60 has aperture 62 positioned above exposed portion 106e. Aperture 62 is a ventilation hole leading from the outside to sensor unit 100 and penetrates molded resin portion 60 in the thickness direction of molded resin portion 60. Aperture 62 is formed using protrusion 92 protruding from the inner side of mold 90. When the shape of protrusion 92 is conical or cylindrical, for example, the shape of edge 62e of aperture 62 is circular.

In the present variation, when molded resin portion 60 is formed, edge 92e of protrusion 92 of mold 90 is pressed against flat part 47 of protective film 45 covering convex portion 36. This results in sensor package 10G after molding having a structure in which edge 62e of aperture 62 contacts flat part 47 of protective film 45 and is formed along flat part 47 of protective film 45. Stated differently, edge 62e of aperture 62 is positioned in the same plane on the surface 31 side of chip 30.

According to the above-described structure, when molded resin portion 60 is formed, for example, it is unlikely that a gap is formed between protrusion 92 of mold 90 and flat parts 47 of chip 30. Specifically, since chip 30 includes one or more annular flat parts 47, when molded resin portion 60 is formed, the bottom of protrusion 92 contacts flat parts 47 and leaking out of the resin material toward sensor unit 100 can be inhibited. This can suppress deterioration in the quality of sensor unit 100. Furthermore, with two or more circular patterns, it is possible to trap resin that has intruded into the annular concave portions (opening grooves) between the plurality of convex portions 36 even when the resin intrudes beyond the outer circular pattern. For this reason, it is possible to reduce the risk that sensor unit 100 is covered with resin even beyond the innermost circular pattern.

Although the above has illustrated an example in which the inner circumference of flat part 47 of chip 30 is circular in shape, the shape of flat part 47 is not limited to this example. The inner circumference of flat part 47 of chip 30 may be angular.

Although the above has illustrated an example in which convex portion 36 is formed due to annular dummy electrode 34 below flat part 47 of protective film 45, the formation of convex portion 36 is not limited to this example. For example, dummy electrode 34 for forming convex portion 36 may be absent or may be a non-circular pattern (e.g., wiring for electrical connection or an island-like dummy pattern).

### Variation 8 of Embodiment 1

Sensor package 10H according to Variation 8 of Embodiment 1 will be described. In Variation 8, an example in which the sensor package according to Variation 4 is provided with guiding grooves 48 that guide the flows of resin material will be described.

Sensor package 10H according to Variation 8 includes substrate 20, chip 30 mounted on substrate 20, and molded resin portion 60 provided on chip 30 and substrate 20. The configurations of substrate 20 and molded resin portion 60 are the same as those described in Variation 4.

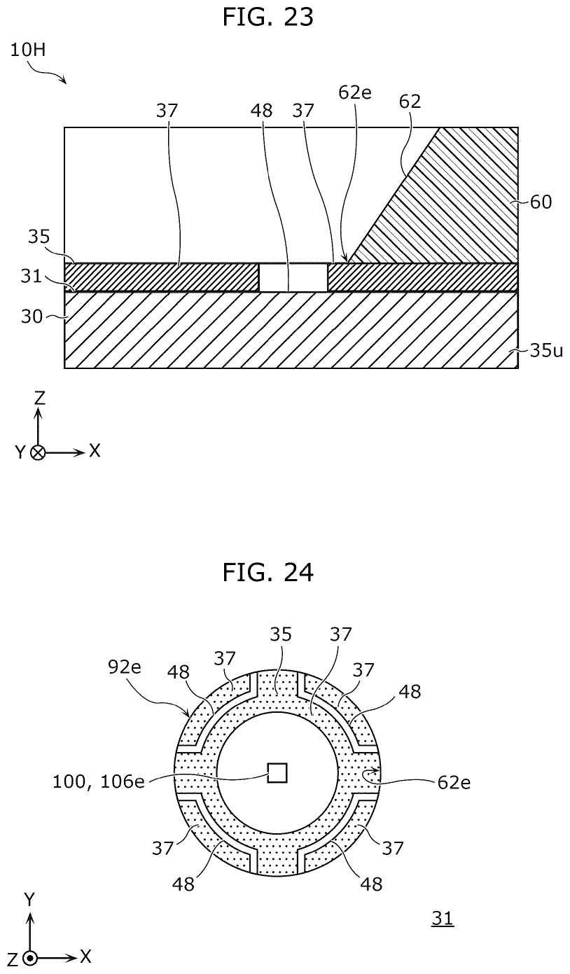

FIG. 23 is a diagram illustrating a cross section of chip 30 and molded resin portion 60 of sensor package 10H according to Variation 8.

Chip 30 has multiple insulating films. Topmost insulating film 35 is formed on lower insulating film 35u that is flattened. Topmost insulating film 35 includes flat parts 37 that are flat areas and guiding grooves 48 that are recessed with respect to flat parts 37. Guiding grooves 48 are grooves to guide resin material that has intruded between protrusion 92 and flat parts 37 at the time of resin molding in a direction different from the direction in which exposed portion 106e is located. Guiding grooves 48 penetrate insulating film 35 in the thickness direction of insulating film 35 and are formed so that lower insulating film 35u is exposed. Guiding grooves 48 in this example are formed in one circle, but may be formed in two or more circles. For example, the amount of resin that can be trapped can be increased by lengthening the circumferential path of guiding groove 48 by using a spiral or snake shape.

FIG. 24 is a diagram illustrating guiding grooves 48 of chip 30 according to Variation 8 of Embodiment 1.

Guiding groove 48 illustrated in FIG. 24 is formed to guide resin material in the circumferential direction. Guiding groove 48 is arcuate in shape and is formed along the inner side face of the outermost flat part 37. A plurality of guiding grooves 48 are formed in flat parts 37. One end of arcuate guiding groove 48 is connected to a predetermined location on edge 62e of aperture 62, and the other end of arcuate guiding groove 48 is connected to other location on edge 62e of aperture 62. Both of the one end and the other end of guiding groove 48 do not need to contact edge 62e and one of these ends may be positioned inside of edge 62e. For example, one end of guiding groove 48 may be connected to edge 62e and the other edge may be positioned inside of edge 62e. One end of guiding groove 48 may be positioned inside of edge 62e and the other end may be connected to edge 62e. Only one of the one end and the other end of guiding groove 48 does not need to be positioned inside of edge 62e and both of the one end and the other end may be positioned inside of edge 62e.

In the present variation, when molded resin portion 60 is formed, edge 92e of protrusion 92 of mold 90 is pressed against the outermost flat part 37. This results in sensor package 10H after molding having a structure in which edge 62e of aperture 62 contacts the outermost flat part 37 and is formed along the outermost flat part 37.

According to the above-described structure, when molded resin portion 60 is formed, for example, it is unlikely that a gap is formed between protrusion 92 of mold 90 and flat parts 37 of chip 30. For this reason, it is possible to inhibit resin material for forming molded resin portion 60 from intruding toward sensor unit 100. This can suppress deterioration in the quality of sensor unit 100.

According to the above-described structure, even when resin material for forming molded resin portion 60 intrudes between flat parts 37 and protrusion 92, for example, it is possible to guide the resin material along guiding grooves 48 of flat parts 37 and stop the intrusion of the resin material inside of guiding grooves 48. This can suppress deterioration in the quality of sensor unit 100.

Although the above has illustrated an example in which the shape of guiding groove 48 is arcuate, the shape of guiding groove 48 is not limited to this example.

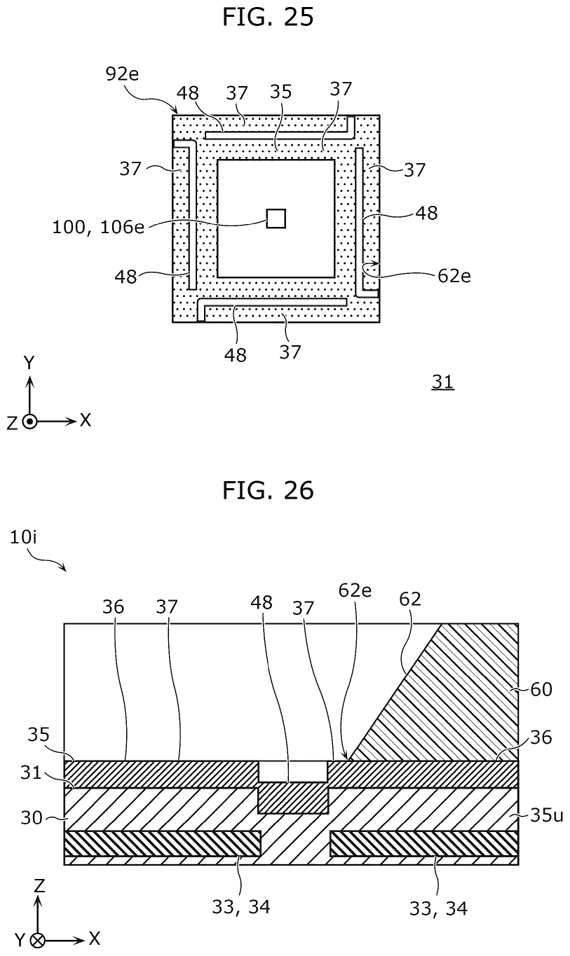

FIG. 25 is a diagram illustrating another example of guiding groove 48 of chip 30.

As illustrated in the figure, guiding groove 48 of chip 30 may be of a shape having four sides. When guiding groove 48 is formed to have four sides, the shape of protrusion 92 of mold 90 may be quadrangular and the shape of edge 62e of aperture 62 may be angular.

### Variation 9 of Embodiment 1

Sensor package 10i according to Variation 9 of Embodiment 1 will be described. In Variation 9, an example in which another insulating film is formed between dummy electrode 34 and convex portion 36 in the sensor package according to Variation 8 will be described.

Sensor package 10i according to Variation 9 includes substrate 20, chip 30 mounted on substrate 20, and molded resin portion 60 provided on chip 30 and substrate 20. The configurations of substrate 20 and molded resin portion 60 are the same as those described in Variation 8.

FIG. 26 is a diagram illustrating a cross section of chip 30 and molded resin portion 60 of sensor package 10i according to Variation 9.

Chip 30 includes multiple wirings and multiple insulating films. Dummy electrode 34 has a predetermined thickness and is formed annularly outside of exposed portion 106e. In the figure, an example in which wiring 33 is composed of dummy electrode 34 is illustrated, but wiring 33 is not limited to this example. For example, wiring 33 may be composed of a combination of a dummy electrode and wiring for electrical connection. Moreover, wiring 33 does not need to be a closed ring, but may be formed so that open locations are provided by multiple wiring patterns. By providing open locations, it is possible to provide a wiring path for electrical connection between the inside and outside of edge 62e.

In Variation 9, lower insulating film 35u is formed over dummy electrode 34, and the topmost insulating film 35 is formed on lower insulating film 35u.

Lower insulating film 35u is provided in chip 30 to cover dummy electrodes 34 each having a predetermined thickness. For this reason, convex portions are formed in lower insulating film 35u in such a manner to correspond to positions in which dummy electrodes 34 are provided. Topmost insulating film 35 is provided on surface 31 of chip 30 to cover the convex portions of lower insulating film 35u. For this reason, convex portions 36 are formed in topmost insulating film 35 in such a manner to correspond to positions in which dummy electrodes 34 and the convex portions of lower insulating film 35u are provided.

In other words, insulating film 35 includes convex portions 36 protruding in a direction perpendicular to surface 31 of chip 30, and convex portions 36 are formed by insulating film 35 covering over dummy electrodes 34. These convex portions 36 are formed annularly to surround exposed portion 106e along the shapes of dummy electrodes 34. The tips of convex portions 36 are flat parts 37.

Insulating film 35 according to Variation 9 includes: flat parts 37 at the tips of convex portions 36; and guiding grooves 48 that are recessed with respect to flat parts 37. Guiding grooves 48 are arcuate in shape and are formed along the inner side face of the outermost flat part 37. A plurality of guiding grooves 48 are formed in flat parts 37. One end of arcuate guiding groove 48 is connected to a predetermined location on edge 62e of aperture 62, and the other end of arcuate guiding groove 48 is connected to other location on edge 62e of aperture 62. Guiding grooves 48 may be formed by forming dummy electrodes 34 in lower insulating film 35u (see FIG. 13) and covering dummy electrodes 34 and lower insulating film 35u with insulating film 35. In other words, guiding grooves 48 may be formed using the difference in height of insulating film 35 caused by presence or absence of dummy electrode 34.

Molded resin portion 60 has aperture 62 positioned above exposed portion 106e. Aperture 62 is a ventilation hole leading from the outside to sensor unit 100 and penetrates molded resin portion 60 in the thickness direction of molded resin portion 60. Aperture 62 is formed using protrusion 92 protruding from the inner side of mold 90. When the shape of protrusion 92 is conical or cylindrical, for example, the shape of edge 62e of aperture 62 is circular.

In the present variation, when molded resin portion 60 is formed, edge 92e of protrusion 92 of mold 90 is pressed against the outermost flat part 37. This results in sensor package 10i after molding having a structure in which edge 62e of aperture 62 contacts the outermost flat part 37 and is formed along the outermost flat part 37.

According to the above-described structure, when molded resin portion 60 is formed, for example, it is unlikely that a gap is formed between protrusion 92 of mold 90 and flat parts 37 of chip 30. For this reason, it is possible to inhibit resin material for forming molded resin portion 60 from intruding toward sensor unit 100. Since chip 30 has two or more annular flat parts 37, it is possible to inhibit the resin material from leaking out toward sensor unit 100. This can suppress deterioration in the quality of sensor unit 100.

According to the above-described structure, even when resin material for forming molded resin portion 60 intrudes between flat parts 37 and protrusion 92, for example, it is possible to guide the resin material along guiding grooves 48 of flat parts 37 and stop the intrusion of the resin material inside of guiding grooves 48. This can suppress deterioration in the quality of sensor unit 100.

Although the above has illustrated an example in which the shape of guiding grooves 48 is arcuate, the shape of guiding grooves 48 is not limited to this example. The shape of guiding grooves 48 of chip 30 may be of a shape having four sides.

### Variation 10 of Embodiment 1

Sensor package 10J according to Variation 10 of Embodiment 1 will be described. In Variation 10, an example in which chip 30 includes two strip-shaped flat parts 37 will be described.

Sensor package 10J according to Variation 10 includes substrate 20, chip 30 mounted on substrate 20, and molded resin portion 60 provided on chip 30 and substrate 20. The configuration of substrate 20 is the same as that described in Embodiment 1.

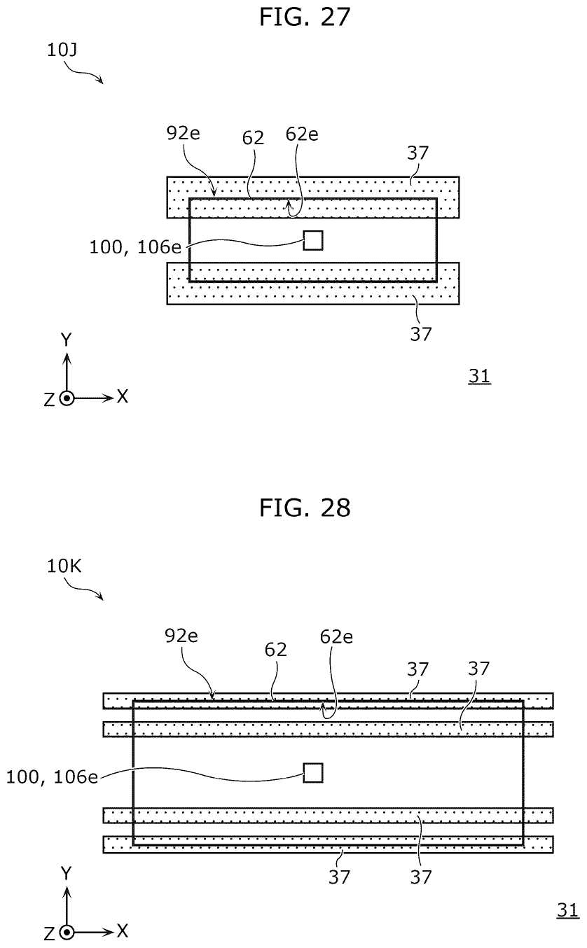

FIG. 27 is a diagram illustrating the top view of flat parts 37 of chip 30 of sensor package 10J according to Variation 10.

Flat part 37 is positioned outside of exposed portion 106e on surface 31 of chip 30. Chip 30 according to Variation 10 has two flat parts 37. Each flat part 37 is strip-shaped when viewed from a direction perpendicular to surface 31 of chip 30. The two strip-shaped flat parts 37 are provided on both sides of exposed portion 106e outside of exposed portion 106e and are parallel to each other.

Molded resin portion 60 has aperture 62 positioned above exposed portion 106e. Aperture 62 is rectangular in shape when viewed from a direction perpendicular to surface 31 of chip 30. The length of long edges 62e of aperture 62 is formed so that the short sides are distant from exposed portion 106e and long enough to ensure that resin entering from the short sides of aperture 62 does not reach exposed portion 106e. The length of long edge 62e is, for example, between three and six times the length of short edge 62e. Two long edges 62e of aperture 62 are respectively formed along the two strip-shaped flat parts 37. In this example, flat parts 37 are not formed in positions corresponding to the two short sides of aperture 62.

Aperture 62 is formed using protrusion 92 protruding from the inner side of mold 90. Protrusion 92 in this example has a prismatic or quadrangular pyramidal shape including the bottom surface with long and short sides.

In the present variation, when molded resin portion 60 is formed, long edge 92e of protrusion 92 of mold 90 is pressed against two strip-shaped flat parts 37. This results in sensor package 10J after molding having a structure in which long edges 62e of aperture 62 contact strip-shaped flat parts 37 and are formed along strip-shaped flat parts 37.

According to the above-described structure, when molded resin portion 60 is formed, for example, it is unlikely that a gap is formed between protrusion 92 of mold 90 and flat parts 37 of chip 30. For this reason, it is possible to inhibit resin material for forming molded resin portion 60 from intruding toward sensor unit 100. This can suppress deterioration in the quality of sensor unit 100.

### Variation 11 of Embodiment 1

Sensor package 10K according to Variation 11 of Embodiment 1 will be described. In Variation 11, an example in which chip 30 includes two sets of double strip-shaped flat parts 37 will be described.

Sensor package 10K according to Variation 11 includes substrate 20, chip 30 mounted on substrate 20, and molded resin portion 60 provided on chip 30 and substrate 20. The configuration of substrate 20 is the same as that described in Embodiment 1.

FIG. 28 is a diagram illustrating the top view of flat parts 37 of chip 30 of sensor package 10K according to Variation 11.

Flat parts 37 are positioned outside of exposed portion 106e on surface 31 of chip 30. Chip 30 according to Variation 11 includes two sets of double strip-shaped flat parts 37. The two sets of double strip-shaped flat parts 37 are provided on both sides of exposed portion 106e outside of exposed portion 106e and are parallel to each other.

Molded resin portion 60 has aperture 62 positioned above exposed portion 106e. Aperture 62 is rectangular in shape when viewed from a direction perpendicular to surface 31 of chip 30. The length of long edge 62e of aperture 62 is, for example, between three and six times the length of short edge 62e. Each of two long edges 62e of aperture 62 is formed along one strip-shaped flat part 37 out of the double strip-shaped flat parts 37. In this example, flat parts 37 are not formed in the positions corresponding to the two short sides of aperture 62.

Aperture 62 is formed using protrusion 92 protruding from the inner side of mold 90. Protrusion 92 in this example has a prismatic or quadrangular pyramidal shape with long and short sides.

In the present variation, when molded resin portion 60 is formed, long edge 92e of protrusion 92 of mold 90 is pressed against strip-shaped flat parts 37. This results in sensor package 10K after molding having a structure in which part of edge 62e of aperture 62 contacts strip-shaped flat parts 37 and is formed along strip-shaped flat parts 37.

According to the above-described structure, when molded resin portion 60 is formed, for example, it is unlikely that a gap is formed between protrusion 92 of mold 90 and flat parts 37 of chip 30. For this reason, it is possible to inhibit resin material for forming molded resin portion 60 from intruding toward sensor unit 100. This can suppress deterioration in the quality of sensor unit 100.

### Variation 12 of Embodiment 1

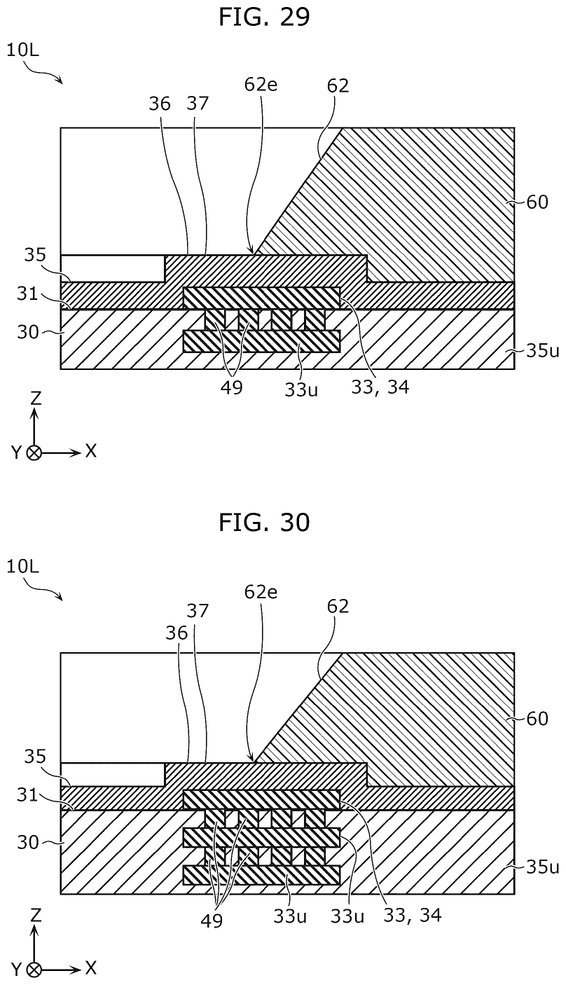

Sensor package 10L according to Variation 12 of Embodiment 1 will be described with reference to FIG. 29 to FIG. 31. In Variation 12, an example in which chip 30 has a crack prevention structure will be described.

Sensor package 10L according to Variation 12 includes substrate 20, chip 30 mounted on substrate 20, and molded resin portion 60 provided on chip 30 and substrate 20. The configurations of substrate 20 and molded resin portion 60 are the same as those described in Embodiment 1.

FIG. 29 is a diagram illustrating a cross section of via conductors 49 of chip 30 of sensor package 10L according to Variation 12.

As illustrated in the figure, topmost wiring 33 is connected to lower wiring 33u via a plurality of via conductors 49. This can increase strength below insulating film 35 subjected to pressing force from mold 90. According to this structure, defects such as cracks in chip 30 can be inhibited. This can suppress deterioration in the quality of sensor unit 100.

FIG. 30 is a diagram illustrating another example of a cross section of via conductors 49.

As illustrated in the figure, a plurality of via conductors 49 may be formed in a multilayer structure.

FIG. 31 is the top views of via conductors 49.

The plurality of via conductors 49 may be arranged in a matrix as illustrated in (a) in FIG. 31 or staggered alternately as illustrated in (b). The plurality of via conductors 49 may be formed in a honeycomb shape as illustrated in (c) in FIG. 31 or in multiple lines as illustrated in (d).

### Embodiment 2

### [Configuration of Sensor Package]

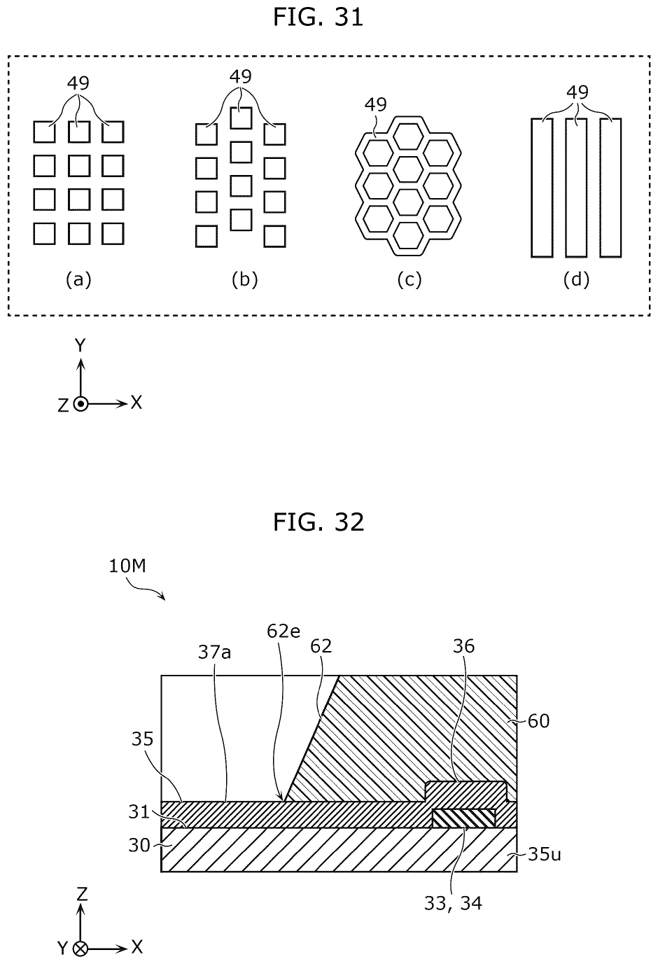

Sensor package 10M according to Embodiment 2 will be described with reference to FIG. 32 to FIG. 34. In Embodiment 2, an example in which edge 62e of aperture 62 is positioned more inside relative to convex portion 36 of insulating film 35 will be described.

Sensor package 10M includes substrate 20, chip 30 mounted on substrate 20, and molded resin portion 60 provided on chip 30 and substrate 20. The configurations of substrate 20 and chip 30 are the same as those described in Embodiment 1.

FIG. 32 is a diagram illustrating a cross section of chip 30 and molded resin portion 60 of sensor package 10M.

Chip 30 includes multiple insulating wirings and multiple insulating films. In this example, topmost wirings 33 are formed on lower insulating film 35u that is flattened. For example, some of topmost wirings 33 are each composed of dummy electrode 34 that is not electrically connected to sensor unit 100. Dummy electrode 34 has a predetermined thickness and is formed annularly outside of exposed portion 106e. Dummy electrode 34 in this example is annular in shape.

Topmost insulating film 35 is formed on lower insulating film 35u and topmost wirings 33. In this example, the insulating film formed on surface 31 of chip 30 is topmost insulating film 35. The material of topmost insulating film 35 is, for example, silicon nitride.

Insulating film 35 is provided on surface 31 of chip 30 to cover dummy electrode 34 having the predetermined thickness. For this reason, convex portion 36 is formed in insulating film 35 in such a manner to correspond to a position in which dummy electrode 34 is provided. In other words, insulating film 35 includes convex portion 36 protruding in a direction perpendicular to surface 31 of chip 30, and convex portion 36 is formed by insulating film 35 covering over dummy electrode 34. This convex portion 36 is formed annularly to surround exposed portion 106e along the shape of dummy electrode 34.

In Embodiment 2, flat part 37a is provided more inside relative to annular convex portion 36. Flat part 37a in this example is formed to be flat by not placing upper or lower wiring that causes a step below flat part 37a. Flat part 37a positioned more inside relative to convex portion 36 and formed continuously flat without any steps in the circumferential direction that surrounds exposed portion 106e. The difference in height of irregularities of flat part 37a is, for example, less than 1 μm.

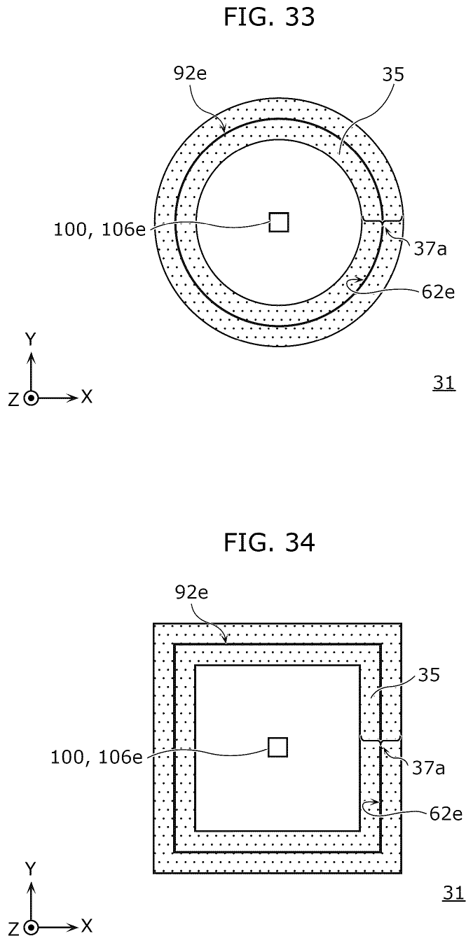

FIG. 33 is the top view of flat part 37a of chip 30 of sensor package 10M.

Flat part 37a is positioned outside of exposed portion 106e on surface 31 of chip 30. Flat part 37a is formed annularly to surround exposed portion 106e. Flat part 37a in this example is annular in shape.

Molded resin portion 60 is formed by molding thermosetting resin. Molded resin portion 60 is provided above chip 30 and substrate 20 not to cover exposed portion 106e of sensor unit 100. In other words, molded resin portion 60 is formed to cover surface 21 of substrate 20 and surface 31 of chip 30 excluding exposed portion 106e.

Molded resin portion 60 has aperture 62 positioned above exposed portion 106e. Aperture 62 is a ventilation hole leading from the outside to sensor unit 100 and penetrates molded resin portion 60 in the thickness direction of molded resin portion 60. In this example, a single aperture 62 is formed at the center of molded resin portion 60. Aperture 62 is formed in a tapered shape so that the area of the hole increases from the inside toward the outside. Aperture 62 is formed using protrusion 92 protruding from the inner side of mold 90. When the shape of protrusion 92 is conical or cylindrical, for example, the shape of edge 62e of aperture 62 is circular.

In the present embodiment, when molded resin portion 60 is formed, edge 92e of protrusion 92 of mold 90 is pressed against flat part 37a positioned more inside relative to convex portion 36. This results in sensor package 10M after molding having a structure in which edge 62e of aperture 62 on the surface 31 side of chip 30 contacts flat part 37a of insulating film 35 and is formed along flat part 37a that is flat in the circumferential direction. Stated differently, edge 62e of aperture 62 is positioned in the same plane on the surface 31 side of chip 30.

According to the above-described structure, when molded resin portion 60 is formed, for example, it is unlikely that a gap is formed between protrusion 92 of mold 90 and flat part 37a of chip 30. For this reason, it is possible to inhibit resin material for forming molded resin portion 60 from intruding toward sensor unit 100. This can suppress deterioration in the quality of sensor unit 100.

According to the above-described structure, since intrusion of the resin material can be inhibited, the pressing force of mold 90 against chip 30 can be reduced and the occurrence of cracks in chip 30 can be inhibited. Since the intrusion of the resin material can be inhibited, the sizes of aperture 62 and chip 30 can be reduced.

Although the above has illustrated an example in which the shape of flat part 37a of chip 30 is annular, the shape of flat part 37a is not limited to this example.

FIG. 34 is a diagram illustrating another example of flat part 37a of chip 30.

As illustrated in the figure, the shape of flat part 37a of chip 30 may be angular annular. The angles in this example are square or rectangular in shape. When the shape of flat part 37a is angular annular, the shape of convex portion 36 of insulating film 35 as well as the shape of dummy electrode 34 for forming convex portion 36 may be also angular annular. The shape of protrusion 92 of mold 90 may be pyramidal or prismatic and the shape of edge 62e of aperture 62 may be angular.

Moreover, the above has illustrated an example in which dummy electrode 34 and convex portion 36 are formed annularly, but the formation of dummy electrode 34 and convex portion 36 is not limited to this example. Dummy electrode 34 and convex portion 36 according to Embodiment 2 may be scattered in plural positions and arranged like islands if formed outside of flat part 37a. The above has illustrated an example of forming convex portion 36 by covering, with insulating film 35, only dummy electrode 34, but the formation of convex portion 36 is not limited to this example. For example, convex portion 36 may be formed by covering, with insulating film 35, wiring in which a dummy electrode and wiring for electric connection are combined.

### Variation 1 of Embodiment 2

Sensor package 10N according to Variation 1 of Embodiment 2 will be described. In Variation 1 of Embodiment 2, an example in which lower wiring 33u is formed in lower insulating film 35u, the surface of lower insulating film 35u on the upper side of lower wiring 33u is flattened, and insulating film 35 provided thereon is flat will be described.

Sensor package 10N according to Variation 1 of Embodiment 2 includes substrate 20, chip 30 mounted on substrate 20, and molded resin portion 60 provided on chip 30 and substrate 20. The configurations of substrate 20 and molded resin portion 60 are the same as those described in Embodiment 2.

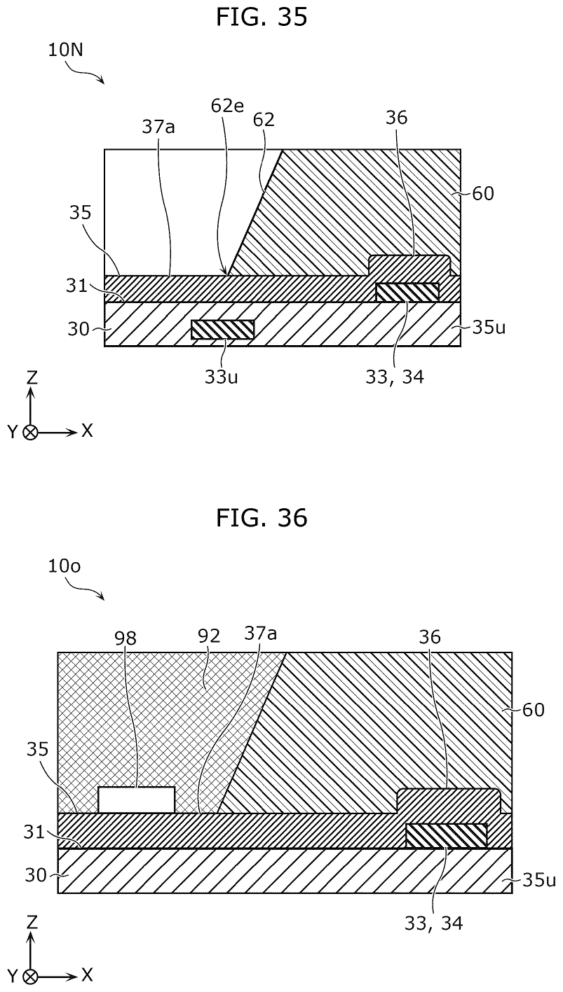

FIG. 35 is a diagram illustrating a cross section of chip 30 and molded resin portion 60 of sensor package 10N.

Chip 30 includes multiple insulating wirings and multiple insulating films. Dummy electrode 34 has a predetermined thickness and is formed annularly outside of exposed portion 106e.

In Variation 1, lower wiring 33u is formed in a position that is lower and more inside relative to dummy electrode 34, and lower insulating film 35u is formed to cover this lower wiring 33u. A flattening process is performed on lower insulating film 35u. For this reason, the surface of lower insulating film 35u is flat.

Flat part 37a, which is a flat area, is provided more inside relative to convex portion 36 of insulating film 35. Flat part 37a positioned more inside relative to convex portion 36 is formed continuously flat without any steps in the circumferential direction that surrounds exposed portion 106e.

Flat part 37a is positioned outside of exposed portion 106e on surface 31 of chip 30. Flat part 37a is formed annularly to surround exposed portion 106e. Flat part 37a in this example is annular in shape.

Molded resin portion 60 has aperture 62 positioned above exposed portion 106e. Aperture 62 is a ventilation hole leading from the outside to sensor unit 100 and penetrates molded resin portion 60 in the thickness direction of molded resin portion 60. Aperture 62 is formed using protrusion 92 protruding from the inner side of mold 90. When the shape of protrusion 92 is conical or cylindrical, for example, the shape of edge 62e of aperture 62 is circular.

In the present variation, when molded resin portion 60 is formed, edge 92e of protrusion 92 of mold 90 is pressed against flat part 37a positioned more inside relative to convex portion 36. This results in sensor package 10N after molding having a structure in which edge 62e of aperture 62 contacts flat part 37a of insulating film 35 and is formed along flat part 37a that is flat in the circumferential direction. Stated differently, edge 62e of aperture 62 is positioned in the same plane on the surface 31 side of chip 30. With this structure, the same advantageous effects as those described in Embodiment 2 can be achieved.

The above has illustrated an example in which convex portion 36 is formed by covering only dummy electrode 34 with insulating film 35, but the formation of concave portion 36 is not limited to this example. For example, convex portion 36 may be formed by covering, with insulating film 35, wiring in which a dummy electrode and wiring for electrical connection are combined.

### Variation 2 of Embodiment 2

Sensor package 10o according to Variation 2 of Embodiment 2 will be described. In Variation 2 of Embodiment 2, an example in which guiding groove 98 that guides the flow of resin material is provided in protrusion 92 of mold 90 in the sensor package according to Embodiment 2 will be described.

Sensor package 10o according to Variation 2 of Embodiment 2 includes substrate 20, chip 30 mounted on substrate 20, and molded resin portion 60 provided on chip 30 and substrate 20. The configurations of substrate 20, chip 30, and molded resin portion 60 are the same as those described in Embodiment 2.

FIG. 36 is a diagram illustrating a cross section of sensor package 10o and protrusion 92 of mold 90.

As illustrated in the figure, guiding groove 98 that guides resin material is provided on the bottom surface of protrusion 92 of mold 90. Guiding groove 98 is a groove that guides resin material that has intruded between protrusion 92 and flat part 37a at the time of resin molding in a direction different from the direction in which exposed portion 106e is located.

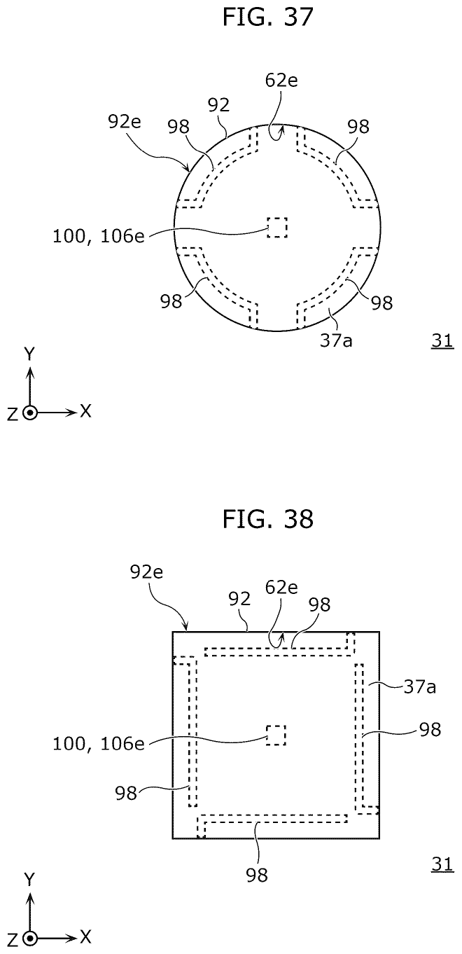

FIG. 37 is a plan view illustrating guiding grooves 98 of protrusion 92 of mold 90. FIG. 37 also illustrates, for reference, surface 31 of chip 30, flat part 37a, edge 62e of aperture 62, sensor unit 100, and exposed portion 106e.

A plurality of guiding grooves 98 illustrated in FIG. 37 are formed on the bottom surface of protrusion 92. Guiding grooves 98 are formed to guide resin material in the circumferential direction. Guiding grooves 98 are arcuate in shape and are formed along the side face of protrusion 92. One end of arcuate guiding groove 98 is connected to a predetermined location on the side face of protrusion 92 and the other end of arcuate guiding groove 98 is connected to other location on the side face of protrusion 92. The one end and the other end of guiding groove 98 may not contact the side face of protrusion 92. Even when guiding grooves 98 are formed more inside relative to the side face of protrusion 92, the resin that has intruded can be trapped.

In the present variation, when molded resin portion 60 is formed, edge 92e of protrusion 92 of mold 90 is pressed against flat part 37a positioned more inside relative to convex portion 36. This results in sensor package 10o after molding having a structure in which edge 62e of aperture 62 contacts flat part 37a of insulating film 35 and is formed along flat part 37a that is flat in the circumferential direction. With this structure, the same advantageous effects as those described in Embodiment 2 can be achieved.

According to the above-described structure, even when resin material for forming molded resin portion 60 intrudes between flat part 37a and protrusion 92, for example, it is possible to guide the resin material along guiding grooves 98 of protrusion 92 and stop the intrusion of the resin material inside of guiding grooves 98. This can suppress deterioration in the quality of sensor unit 100.

Although the above has illustrated an example in which the shape of guiding grooves 98 is arcuate, the shape of guiding grooves 98 is not limited to this example.

FIG. 38 is a diagram illustrating another example of guiding grooves 98 of protrusion 92 of mold 90. FIG. 38 also illustrates, for reference, surface 31 of chip 30, flat part 37a, edge 62a of aperture 62, sensor unit 100, and exposed portion 106e.

As illustrated in the figure, guiding groove 98 of protrusion 92 may have a shape having four sides. When guiding groove 98 is formed to have four sides, the shape of protrusion 92 of mold 90 may be quadrangular pyramidal or quadrangular columnar.

### [Sensor Package Manufacturing Method]

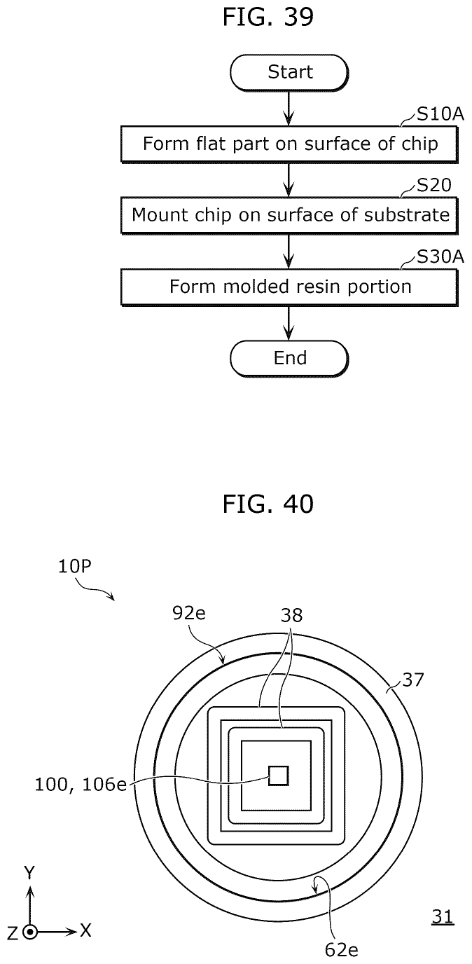

A method of manufacturing sensor package 10M according to Embodiment 2 will be described with reference to FIG. 39.

FIG. 39 is a flowchart illustrating the method of manufacturing sensor package 10M.

The manufacturing method of sensor package 10M illustrated in FIG. 39 includes step S10A of forming flat part 37a on surface 31 of chip 30, step S20 of mounting chip 30 on surface 21 of substrate 20, and step S30A of forming molded resin portion 60.

First, in step S10A, flat part 37a is provided outside of an area in which exposed portion 106e of sensor unit 100 is formed. Flat part 37a may be provided in chip 30 before or after exposed portion 106e is formed.

### Subsequently in Step S20, Chip 30 Is Mounted on Substrate 20 by wire bonding. With this, chip 30 and substrate 20 are electrically connected to each other via wires 50.

Subsequently in step S30A, protrusion 92 of mold 90 is arranged above exposed portion 106e of sensor unit 100 of chip 30, and molded resin portion 60 is formed to cover surface 21 of substrate 20 and surface 31 of chip 30 excluding exposed portion 106e. In this example, mold 90 is arranged so that edge 92e of protrusion 92 is positioned on flat part 37a, to mold resin. When the resin is molded, a film-like resin sheet is placed along the inner face of mold 90 to form molded resin portion 60.

Moreover, in step S30A, the resin may be molded using protrusion 92 of mold 90 including guiding grooves 98 that guide the flows of resin material of molded resin portion 60.

Thus, when molded resin portion 60 is formed, by arranging edge 92e of protrusion 92 of mold 90 on flat part 37a, it is possible to inhibit a gap from being formed between protrusion 92 and flat part 37a. For this reason, it is possible to inhibit resin material for forming molded resin portion 60 from intruding toward sensor unit 100. This can suppress deterioration in the quality of sensor unit 100.

### Variations of Embodiment 1 and Embodiment 2

FIG. 40 is the top view of flat part 37 of chip 30 of sensor package 10P according to a variation of Embodiment 1 and Embodiment 2.

As illustrated in the figure, sensor package 10P may include annular flat part 37 outside of exposed portion 106e of sensor unit 100. Angular opening grooves 38 may be provided between exposed portion 106e and annular flat part 37. Angular opening grooves 38 may be provided double. With this structure, the same advantageous effects as those described in Embodiments 1 and 2 can be achieved.

### Conclusion

Sensor packages 10 to 10P according to one aspect of the present disclosure will be illustrated.

A sensor package according to Example 1 includes: chip 30 including surface 31 on which exposed portion 106e of sensor unit 100 is provided; substrate 20 including surface 21 on which chip 30 is mounted; and molded resin portion 60 covering surface 21 of substrate 20 and surface 31 of chip 30 excluding exposed portion 106e. Molded resin portion 60 includes aperture 62 positioned above exposed portion 106e. Chip 30 includes a flat part (37, 37a, or 47) positioned outside of exposed portion 106e on surface 31 of chip 30. Edge 62e of aperture 62 on the surface 31 side of chip 30 is formed along the flat part.

Thus, with a structure in which edge 62e of aperture 62 is formed along the flat part, it is unlikely that a gap is formed between protrusion 92 of mold 90 for forming aperture 62 and the flat part of chip 30 when molded resin portion 60 is formed, for example. It is therefore possible to inhibit resin material for forming molded resin portion 60 from intruding toward sensor unit 100. This can suppress deterioration in the quality of sensor unit 100.

A sensor package according to Example 2 is the sensor package according to Example 1, and the flat part (37, 37a, or 47) may be formed annularly to surround exposed portion 106e.

According to this configuration, when molded resin portion 60 is formed, for example, it is unlikely that a gap is formed at the entire circumference that surrounds exposed portion 106e. It is therefore possible to inhibit resin material for forming molded resin portion 60 from intruding toward sensor unit 100. This can suppress deterioration in the quality of sensor unit 100.

A sensor package according to Example 3 is the sensor package according to Example 1 or 2, and chip 30 includes insulating film 35 on surface 31 of chip 30. Insulating film 35 includes convex portion 36 protruding in a direction perpendicular to surface 31 of chip 30. The flat part (37 or 47) may be provided on convex portion 36.

According to this configuration, when molded resin portion 60 is formed, for example, protrusion 92 of mold 90 can be pressed against the flat part on convex portion 36 and a gap is unlikely to be formed between protrusion 92 and the flat part. It is therefore possible to inhibit resin material for forming molded resin portion 60 from intruding toward sensor unit 100. This can suppress deterioration in the quality of sensor unit 100.

A sensor package according to Example 4 is the sensor package according to Example 3, and edge 62e of aperture 62 may be positioned on convex portion 36.

According to this configuration, when molded resin portion 60 is formed, for example, edge 92e of protrusion 92 of mold 90 can be pressed against the flat part on convex portion 36 and a gap is unlikely to be formed between protrusion 92 and the flat part. It is therefore possible to inhibit resin material for forming molded resin portion 60 from intruding toward sensor unit 100. This can suppress deterioration in the quality of sensor unit 100.

A sensor package according to Example 5 is the sensor package according to Example 3, and edge 62e of aperture 62 may be positioned outside of convex portion 36.

According to this configuration, when molded resin portion 60 is formed, for example, the bottom surface of protrusion 92 can be pressed against the flat part on convex portion 36 and a gap is unlikely to be formed between protrusion 92 and the flat part. It is therefore possible to inhibit resin material for forming molded resin portion 60 from intruding toward sensor unit 100. This can suppress deterioration in the quality of sensor unit 100.

A sensor package according to Example 6 is the sensor package according to any one of Examples 3 to 5, and chip 30 may include dummy electrode 34 that is not electrically connected to sensor unit 100. Convex portion 36 may be formed by insulating film 35 covering over dummy electrode 34.

According to this configuration, the flat part can be easily formed on convex portion 36. It is therefore possible to readily suppress deterioration in the quality of sensor unit 100.

A sensor package according to Example 7 is the sensor package according to Example 6, and chip 30 may include a plurality of via conductors 49 connected to dummy electrode 34.

According to this configuration, the strength of chip 30 can be increased. This can suppress deterioration in the quality of sensor unit 100.

A sensor package according to Example 8 is the sensor package according to any one of Examples 1 to 7, and chip 30 may include two or more annular flat parts (37, 37a, or 47), each of which is the flat part.

According to this configuration, when molded resin portion 60 is formed, for example, it is possible to further inhibit resin material from intruding toward sensor unit 100. This can suppress deterioration in the quality of sensor unit 100.

A sensor package according to Example 9 is the sensor package according to any one of Examples 1 to 8, and the flat part (37, 37a, or 47) may include guiding groove 48 that guides a flow of resin material of molded resin portion 60.

According to this configuration, when molded resin portion 60 is formed, for example, it is possible to guide resin material that has intruded between protrusion 92 and the flat part in a direction different from the direction in which sensor unit 100 is located. This can suppress deterioration in the quality of sensor unit 100.

A sensor package according to Example 10 is the sensor package according to Example 1, and chip 30 may include two flat parts (37, 37a, or 47) each of which is the flat part. Aperture 62 may be rectangular in shape when viewed from a direction perpendicular to surface 31 of chip 30. Two long sides of aperture 62 may be respectively formed along the two flat parts.

According to this configuration, when molded resin portion 60 is formed, for example, a gap is unlikely to be formed in an area corresponding to the longer-side direction of aperture 62. It is therefore possible to inhibit resin material for forming molded resin portion 60 from intruding toward sensor unit 100. This can suppress deterioration in the quality of sensor unit 100.

A sensor package according to Example 11 is the sensor package according to Example 1, and chip 30 includes insulating film 35 on surface 31 of chip 30. Insulating film 35 includes convex portion 36 protruding in a direction perpendicular to surface 31 of chip 30. Flat part 37a may be provided between exposed portion 106e and convex portion 36.

According to this configuration, when molded resin portion 60 is formed, for example, a gap is unlikely to be formed between protrusion 92 of mold 90 and flat part 37a. It is therefore possible to inhibit resin material for forming molded resin portion 60 from intruding toward sensor unit 100. This can suppress deterioration in the quality of sensor unit 100.

A sensor package according to Example 12 includes: chip 30 including surface 31 on which exposed portion 106e of sensor unit 100 is provided; substrate 20 including surface 21 on which chip 30 is mounted; and molded resin portion 60 covering surface 21 of substrate 20 and surface 31 of chip 30 excluding exposed portion 106e. Molded resin portion 60 includes aperture 62 positioned above exposed portion 106e. Edge 62e of aperture 62 on the surface 31 side of chip 30 is positioned on the same plane on the surface 31 side of chip 30.

Thus, with the structure in which edge 62e of aperture 62 is positioned in the same plane that is surface 31 of chip 30, when molded resin portion 60 is formed, for example, a gap is unlikely to be formed between protrusion 92 of mold 90 and the same plane of chip 30. It is therefore possible to inhibit resin material for forming molded resin portion 60 from intruding toward sensor unit 100. This can suppress deterioration in the quality of sensor unit 100.

A sensor package according to Example 13 is the sensor package according to Example 12, and chip 30 includes a flat part (37, 37a, or 47) positioned outside of exposed portion 106e on surface 31 of chip 30. Edge 62e of aperture 62 may be positioned on the flat part.

According to the structure in which edge 62e of aperture 62 is positioned on the flat part, when molded resin portion 60 is formed, for example, a gap is unlikely to be formed between protrusion 92 of mold 90 and the flat part of chip 30. It is therefore possible to inhibit resin material for forming molded resin portion 60 from intruding toward sensor unit 100. This can suppress deterioration in the quality of sensor unit 100.

A sensor package manufacturing method according to Example 14 includes: forming a flat part (37, 37a, or 47) on surface 31 of chip 30; mounting chip 30 on surface 21 of substrate 20; and arranging protrusion 92 of mold 90 above exposed portion 106e of sensor unit 100 of chip 30, and forming molded resin portion 60 to cover surface 21 of substrate 20 and surface 31 of chip 30 excluding exposed portion 106e. In the forming of the flat part, the flat part is provided outside of an area where exposed portion 106e is formed. In the forming of molded resin portion 60, mold 90 is arranged so that edge 92e of protrusion 92 is positioned on the flat part, to form molded resin portion 60.

By thus arranging edge 92e of protrusion 92 of mold 90 on the flat part when forming molded resin portion 60, it is possible to inhibit a gap from being formed between protrusion 92 and the flat part. It is therefore possible to inhibit resin material for forming molded resin portion 60 from intruding toward sensor unit 100. This can suppress deterioration in the quality of sensor unit 100.

A sensor package manufacturing method according to Example 15 is the sensor package manufacturing method according to Example 14, and in the forming of molded resin portion 60, resin may be molded using protrusion 92 including guiding groove 98 that guides a flow of resin material of molded resin portion 60.

According to this configuration, when molded resin portion 60 is formed, for example, it is possible to guide resin material that has intruded between protrusion 92 and the flat part in a direction different from the direction in which sensor unit 100 is located. This can suppress deterioration in the quality of sensor unit 100.

### Other Embodiments

Although a sensor package and a sensor package manufacturing method according to the present disclosure have been described based on embodiments and variations thereof, the present disclosure is not limited to these embodiments and variations. Other embodiments obtained by various modifications to the embodiments and variations that may be conceived by persons skilled in the art, as well as embodiments resulting from combinations of elements from different embodiments and variations that do not depart from the essence of the present disclosure may be also included in the range of the present disclosure

### INDUSTRIAL APPLICABILITY

The sensor package according to the present disclosure can be used as, for example, a gas sensor package in fuel cell vehicles.

1. A sensor package comprising:
a chip including a surface on which an exposed portion of a sensor unit is provided;
a substrate including a surface on which the chip is mounted; and
a molded resin portion covering the surface of the substrate and the surface of the chip excluding the exposed portion, wherein
the molded resin portion includes an aperture positioned above the exposed portion,
the chip includes a flat part positioned outside of the exposed portion on the surface of the chip, and
an edge of the aperture on a surface side of the chip is formed along the flat part.

2. The sensor package according to claim 1, wherein the flat part is formed annularly to surround the exposed portion.

3. The sensor package according to claim 1, wherein
the chip includes an insulating film on the surface of the chip,
the insulating film includes a convex portion protruding in a direction perpendicular to the surface of the chip, and
the flat part is provided on the convex portion.

4. The sensor package according to claim 3, wherein
the edge of the aperture is positioned on the convex portion.

5. The sensor package according to claim 3, wherein
the edge of the aperture is positioned outside of the convex portion.

6. The sensor package according to claim 3, wherein
the chip includes a dummy electrode that is not electrically connected to the sensor unit, and
the convex portion is formed by the insulating film covering over the dummy electrode.

7. The sensor package according to claim 6, wherein
the chip includes a plurality of via conductors connected to the dummy electrode.

8. The sensor package according to claim 2, wherein
the chip includes two or more annular flat parts, each of which is the flat part.

9. The sensor package according to claim 1, wherein
the flat part includes a guiding groove that guides a flow of resin material of the molded resin portion.

10. The sensor package according to claim 1, wherein
the chip includes two flat parts each of which is the flat part,
the aperture is rectangular in shape when viewed from a direction perpendicular to the surface of the chip, and
two long sides of the aperture are respectively formed along the two flat parts.

11. The sensor package according to claim 1, wherein
the chip includes an insulating film on the surface of the chip,
the insulating film includes a convex portion protruding in a direction perpendicular to the surface of the chip, and
the flat part is provided between the exposed portion and the convex portion.

12. A sensor package comprising:
a chip including a surface on which an exposed portion of a sensor unit is provided;
a substrate including a surface on which the chip is mounted; and
a molded resin portion covering the surface of the substrate and the surface of the chip excluding the exposed portion, wherein
the molded resin portion includes an aperture positioned above the exposed portion, and
an edge of the aperture on a surface side of the chip is positioned on a same plane on the surface side of the chip.

13. The sensor package according to claim 12, wherein
the chip includes a flat part positioned outside of the exposed portion on the surface of the chip, and
the edge of the aperture is positioned on the flat part.

14. A sensor package manufacturing method comprising:
forming a flat part on a surface of a chip;
mounting the chip on a surface of a substrate; and
arranging a protrusion of a mold above an exposed portion of a sensor unit of the chip, and forming a molded resin portion to cover the surface of the substrate and the surface of the chip excluding the exposed portion, wherein
in the forming of the flat part, the flat part is provided outside of an area where the exposed portion is formed, and
in the forming of the molded resin portion, the mold is arranged so that an edge of the protrusion is positioned on the flat part, to form the molded resin portion.

15. The sensor package manufacturing method according to claim 14, wherein
in the forming of the molded resin portion, resin is molded using the protrusion including a guiding groove that guides a flow of resin material of the molded resin portion.
